SEQUENCE LISTING (101 sequences)

SEQ ID NO: 1 - protein, 132 aa, Bacteriophage Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3-18 - skipped (16 entries)

SEQ ID NO: 19 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 20 - DNA, 36 bp, Artificial Sequence
  oligo PH155
gatcaccatgctactagtaaatcagtcacaccaagg

SEQ ID NO: 21 - DNA, 38 bp, Artificial Sequence
  oligo PH156
ttgaagccttggtgtgactgatttactagtagcatggt

SEQ ID NO: 22 - DNA, 33 bp, Artificial Sequence
  oligo PH157
cttcaataaggaacacacaagcaagatggtaag

SEQ ID NO: 23 - DNA, 33 bp, Artificial Sequence
  oligo PH158
atagcgcttaccatcttgcttgtgtgttcctta

SEQ ID NO: 24 - DNA, 36 bp, Artificial Sequence
  oligo PH159
cgctattgttttatatgtgcttttggcggcggcggc

SEQ ID NO: 25 - DNA, 36 bp, Artificial Sequence
  oligo PH160
gaatgcgccgccgccgccaaaagcacatataaaaca

SEQ ID NO: 26 - DNA, 30 bp, Artificial Sequence
  oligo PH161
gcattctgcctttgcggcggatcccggtgg

SEQ ID NO: 27 - DNA, 28 bp, Artificial Sequence
  oligo PH162
aattccaccgggatccgccgcaaaggca

SEQ ID NO: 28 - DNA, 12 bp, Artificial Sequence
  oligo Uni12
agcaaaagcagg

SEQ ID NO: 29 - DNA, 28 bp, Artificial Sequence
  oligo BM-HA-1
tattcgtctcagggagcaaaagcagggg

SEQ ID NO: 30 - DNA, 35 bp, Artificial Sequence
  oligo BM-NS-890R
atatcgtctcgtattagtagaaacaagggtgtttt

SEQ ID NO: 31 - DNA, 26 bp, Artificial Sequence
  oligoPH163
aaggatcccgacaccatctgcatcgg

SEQ ID NO: 32 - DNA, 19 bp, Artificial Sequence
  oligo PH164
tgctcgagtcagcagccgc

SEQ ID NO: 33 - DNA, 4889 bp, Artificial Sequence
  Plasmid pFastBac1_GP67
atgctactagtaaatcagtcacaccaaggcttcaataaggaacacacaagcaagatggtaagcgctattgttttatatgtgcttttggcggcggcggcgcattctgcctttgcggcggatcccggtggaattcaaaggcctacgtcgacgagctcactagtcgcggccgctttcgaatctagagcctgcagtctcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatcacc

SEQ ID NO: 34 - DNA, 6488 bp, Artificial Sequence
  Plasmid pFastBac1_GP67_HA_PR8
atgctactagtaaatcagtcacaccaaggcttcaataaggaacacacaagcaagatggtaagcgctattgttttatatgtgcttttggcggcggcggcgcattctgcctttgcggcggatcccgacaccatctgcatcggctaccacgccaacaacagcaccgacaccgtggataccgtgctggaaaagaacgtgaccgtgacccacagcgtgaacctgctggaagatagccacaacggcaagctgtgccggctgaaaggcatcgcccccctgcagctgggcaagtgcaacatcgccggctggctgctgggcaaccccgagtgcgaccccctgctgcccgtgcggagctggtcctacatcgtggagacccccaacagcgagaacggcatctgctaccccggcgacttcatcgactacgaggaactgcgggagcagctgtccagcgtgagcagcttcgagcggttcgagatcttccccaaagagagcagctggcccaaccacaacaccaacggcgtgaccgccgcctgtagccacgagggcaagagcagcttctaccggaacctgctgtggctgaccgagaaagagggcagctaccccaagctgaagaacagctacgtgaacaagaaaggcaaggaagtgctggtgctgtggggcatccaccacccccccaactccaaagagcagcagaacctgtaccagaacgagaacgcctacgtgagcgtggtgaccagcaactacaaccggcggttcacccccgagatcgccgagcggcccaaagtgcgggaccaggccggcaggatgaactactactggaccctgctgaagcccggcgacaccatcatcttcgaggccaacggcaacctgatcgcccccatgtacgccttcgccctgagccggggcttcggcagcggcatcatcaccagcaacgccagcatgcacgagtgcaacaccaagtgccagacccccctgggcgccatcaacagcagcctgccctaccagaacatccaccccgtgaccatcggcgagtgccccaagtacgtgcggagcgccaagctgcggatggtgaccggcctgcggaacatccccagcatccagtaccggggcctgttcggcgccatcgccggattcatcgagggcggctggaccggcatgatcgacgggtggtatggctaccaccaccagaatgagcagggcagcggctacgccgccgaccagaagagcacccagaacgccatcaacggcatcaccaacaaggtgaacaccgtgatcgagaagatgaacatccagttcaccgccgtgggcaaagagttcaacaagctggaaaagcggatggaaaacctgaacaagaaggtggacgacggcttcctggacatctggacctacaacgccgagctgctggtgctgctggaaaacgagcggaccctggacttccacgacagcaacgtgaagaatctgtacgagaaggtgaaaagccagctgaagaataacgccaaagagatcggcaacggctgcttcgagttctaccacaagtgcgacaacgagtgcatggaaagcgtgcggaacggcacctacgactaccccaagtacagcgaggaaagcaagctgaaccgggagaaagtggacggcgtggggcgcgccctggtgcccagaggctcccccgggtccggctacatccccgaggcccccagggatggccaggcctacgtgcggaaggacggcgaatgggtgctgctgtccaccttcctgggccaccaccatcaccatcacggcggagctagcggcggctgctgactcgaggcatgcggtaccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatcacc

SEQ ID NO: 35 - DNA, 1494 bp, Influenza A virus
gatcccgccaccctgtgcctgggtcaccacgctgtgcccaacggcaccatcgtcaagaccatcaccaacgaccagatcgaggtcaccaacgctaccgagctggtgcagtcctcctccaccggcgagatctgcgactccccccaccagatcctggacggcgagaactgcaccctgatcgacgctctgctgggtgaccctcagtgcgacggtttccagaacaagaagtgggacctgttcgtcgagcgttccaaggcttactccaactgctacccctacgacgtgcccgactacgcttccctgcgttccctggtggcttcctccggcaccctcgagttcaacaacgagtccttcaactggaccggtgtcacccagaacggcacctcttcctcttgcatccgtggttccaacaactccttcttctcccgtctgaactggctgacccacctgaagttcaagtaccccgctctgaacgtgaccatgcccaacaacgagaagttcgacaagctgtacatctggggtgtccaccaccccggcaccgacaacgaccaaatcttcccctacgctcaggcttccggtcgtatcaccgtgtccaccaagcgttcccagcagaccgtgatccccaacatcggttcccgtccccgtgtgcgtaacatcccctcccgtatctccatctactggaccatcgtgaagcccggcgacatcctgctgatcaactccaccggcaacctgatcgctccccgtggttacttcaagatccgttccggcaagtcctccatcatgcgttccgacgctcccatcggcaagtgcaactccgagtgcatcacccccaacggttccatccccaacgacaagcccttccagaacgtgaaccgtatcacctacggtgcttgcccccgttacgtgaagcagaacaccctgaagctggctaccggcatgcgtaacgtgcccgagaagcagacccgtggtatcttcggtgctatcgctggtttcatcgagaacggctgggagggcatggtggacggctggtacggtttccgtcaccagaactccgagggtatcggccaggctgctgacctgaagtccacccaggctgctatcgaccagatcaacggcaagctgaaccgtctgatcggcaagactaacgagaaattccaccagatcgagaaagagttctccgaggtcgagggtcgtatccaggacctcgagaagtacgtggaggacaccaagatcgacctgtggtcctacaacgccgagctgctggtcgctctcgagaaccagcacaccatcgacctgaccgactccgagatgaacaagctgttcgagaaaaccaagaagcagctgcgcgagaacgctgaggacatgggcaacggctgcttcaagatctaccacaagtgcgacaacgcttgcatcggctccatccgtaacggcacctacgaccacgacgtgtaccgtgacgaggctctgaacaaccgtttccagatcaagggtgtcggg

SEQ ID NO: 36 - DNA, 1527 bp, Influenza A virus
gatcccgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaggg

SEQ ID NO: 37 - DNA, 1527 bp, Influenza A virus
gatcccgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagtgagctcagcatgtccatacctgggaagtccctccttttttagaaatgtggtatggcttatcaaaaagaacagtacatacccaacaataaagaaaagctacaataataccaaccaagaagatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtgggg

SEQ ID NO: 38 - DNA, 1527 bp, Influenza A virus
gatcccgatcaaatttgcattggttaccatgcaaacaactcgacagaacaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccaggggatttcaacgactatgaagaactgaaacacctattgagcagaataaaccattttgagaaaattcagatcatccccaaaagttcttggtcagattatgaagcctcatcaggagtgagctcagcatgtccataccagggaagatcctccttttttagaaatgtggtatggcttatcaaaaagaacaatgcatacccaacaataaagagaagttacaataataccaaccaagaggatcttttggtactgtgggggattcaccatccgaatgatgcggcagagcagataaggctctatcaaaacccaactacctatatttccgttgggacatcaacactaaaccagagattggtaccaaaaatagctactagatctaaggtaaatgggcaaagtggaaggatggagttcttttggacaattttaaaatcgaatgatgcaataaactttgagagtaatggaaatttcattgctccagaatatgcatacaaaattgtcaagaaaggggactcaacaattatgaaaagtgagttggaatatggtaactgcaacaccaaatgtcaaactccaataggggcgataaactccagtatgccattccacaacatccaccctctcaccatcggggaatgccccaaatatgtgaaatcaaacagattagtccttgctactgggctcagaaatagccctcaaggagagagaagaagaagaaagagaggactatttggagctatagcagggtttatagagggaggatggcagggaatggtagatggttggtatgggtaccaccatagcaacgagcaggggagtgggtacgctgcagacaaagaatccactcaaaaggcaatagatggagtcaccaataaggtcaactcgatcattaacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtcagactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaggg

SEQ ID NO: 39 - protein, 502 aa, Influenza A virus
DTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQYRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGV

SEQ ID NO: 40 - protein, 495 aa, Influenza A virus
ATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSSCIRGSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGV

SEQ ID NO: 41 - protein, 506 aa, Influenza A virus
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGV

SEQ ID NO: 42 - protein, 506 aa, Influenza A virus
DQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGV

SEQ ID NO: 43 - protein, 506 aa, Influenza A virus
DQICIGYHANNSTEQVDTIMEKNVTVTHAQDILEKTHNGKLCDLDGVKPLILRDCSVAGWLLGNPMCDEFLNVPEWSYIVEKINPANDLCYPGDFNDYEELKHLLSRINHFEKIQIIPKSSWSDYEASSGVSSACPYQGRSSFFRNVVWLIKKNNAYPTIKRSYNNTNQEDLLVLWGIHHPNDAAEQIRLYQNPTTYISVGTSTLNQRLVPKIATRSKVNGQSGRMEFFWTILKSNDAINFESNGNFIAPEYAYKIVKKGDSTIMKSELEYGNCNTKCQTPIGAINSSMPFHNIHPLTIGECPKYVKSNRLVLATGLRNSPQGERRRRKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGV

SEQ ID NO: 44 - protein, 53 aa, Artificial Sequence
  Protein C-terminus
GRALVPRGSPGSGYIPEAPRDGQAYVRKDGEWVLLSTFLGHHHHHHGGASGGC

SEQ ID NO: 45 - DNA, 50 bp, Artificial Sequence
  Oligo 42-1
aactatagcttaagttcgaagacgtcgacgagctcattaactaatggatc

SEQ ID NO: 46 - DNA, 48 bp, Artificial Sequence
  Oligo 42-2
cattagttaatgagctcgtcgacgtcttcgaacttaagctataggtat

SEQ ID NO: 47 - DNA, 52 bp, Artificial Sequence
  Oligo 42T-1
tcgagcaccaccaccaccaccacggtggttgctaataataattgattaatac

SEQ ID NO: 48 - DNA, 52 bp, Artificial Sequence
  Oligo 42T-2
ctaggtattaatcaattattattagcaaccaccgtggtggtggtggtggtgc

SEQ ID NO: 49 - DNA, 28 bp, Artificial Sequence
  Oligo BM-HA-1
tattcgtctcagggagcaaaagcagggg

SEQ ID NO: 50 - DNA, 35 bp, Artificial Sequence; identical to SEQ ID NO: 30
  SEQ ID NO: 50 Oligo BM-NS-890R

SEQ ID NO: 51 - DNA, 31 bp, Artificial Sequence
  Oligo JA35
gagatcatatgagccataacggcaaactgtg

SEQ ID NO: 52 - DNA, 32 bp, Artificial Sequence
  Oligo JA40
aaaaactcgaggcgcacatatttcgggcattc

SEQ ID NO: 53 - DNA, 30 bp, Artificial Sequence
  Oligo JA37
aatttcatatggcgccgctgcaactgggca

SEQ ID NO: 54 - DNA, 30 bp, Artificial Sequence
  Oligo JA39
tttttctcgagttcatgcatgctcgcgttg

SEQ ID NO: 55 - DNA, 30 bp, Artificial Sequence
  Oligo JA36
tattacatatgaaaggcatcgcgccgctgc

SEQ ID NO: 56 - DNA, 30 bp, Artificial Sequence; identical to SEQ ID NO: 54
  Oligo JA39

SEQ ID NO: 57 - DNA, 30 bp, Artificial Sequence; identical to SEQ ID NO: 55
  Oligo JA36

SEQ ID NO: 58 - DNA, 31 bp, Artificial Sequence
  Oligo JA38
aaaaactcgaggctggtaataatgccgctgc

SEQ ID NO: 59 - DNA, 30 bp, Artificial Sequence; identical to SEQ ID NO: 53
  Oligo JA37

SEQ ID NO: 60 - DNA, 5056 bp, Artificial Sequence
  Plasmid pET-42T(+)
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatggatatcgaattcaagcttctgcagctgctcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 61 - DNA, 5857 bp, Artificial Sequence
  Plasmid pET42T_HA1_PR8_42_310
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgctgctggaagatagccataacggcaaactgtgccgtctgaaaggcatcgcgccgctgcaactgggcaaatgtaacattgcgggctggctgctgggcaatccggaatgcgatccgctgctgccggttcgtagctggagctatattgtggaaaccccgaacagcgaaaacggcatttgctatccgggcgatttcatcgattatgaagaactgcgtgaacagctgtctagcgtgagcagctttgaacgctttgaaatcttcccgaaagaaagcagctggccgaaccataacaccaacggcgtgaccgcggcgtgtagccatgaaggcaaaagcagcttttatcgtaacctgctgtggctgaccgaaaaagaaggcagctatccgaaactgaaaaacagctacgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgtcgttttaccccggaaattgcggaacgtccgaaagtgcgtgatcaggcgggtcgtatgaactattattggaccctgctgaaaccgggcgataccattatttttgaagcgaacggcaacctgattgcgccgatgtatgcgtttgccctgagccgtggctttggcagcggcattattaccagcaacgcgagcatgcatgaatgcaacaccaaatgccagaccccgctgggcgcgattaatagcagcctgccgtatcagaacattcatccggtgaccattggcgaatgcccgaaatatgtgcgcctcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 62 - DNA, 5845 bp, Artificial Sequence
  Plasmid pET42T_HA1_PR8_46_310
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagccataacggcaaactgtgccgtctgaaaggcatcgcgccgctgcaactgggcaaatgtaacattgcgggctggctgctgggcaatccggaatgcgatccgctgctgccggttcgtagctggagctatattgtggaaaccccgaacagcgaaaacggcatttgctatccgggcgatttcatcgattatgaagaactgcgtgaacagctgtctagcgtgagcagctttgaacgctttgaaatcttcccgaaagaaagcagctggccgaaccataacaccaacggcgtgaccgcggcgtgtagccatgaaggcaaaagcagcttttatcgtaacctgctgtggctgaccgaaaaagaaggcagctatccgaaactgaaaaacagctacgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgtcgttttaccccggaaattgcggaacgtccgaaagtgcgtgatcaggcgggtcgtatgaactattattggaccctgctgaaaccgggcgataccattatttttgaagcgaacggcaacctgattgcgccgatgtatgcgtttgccctgagccgtggctttggcagcggcattattaccagcaacgcgagcatgcatgaatgcaacaccaaatgccagaccccgctgggcgcgattaatagcagcctgccgtatcagaacattcatccggtgaccattggcgaatgcccgaaatatgtgcgcctcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 63 - DNA, 5707 bp, Artificial Sequence
  Plasmid pET42T_HA1_PR8_57_276
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatggcgccgctgcaactgggcaaatgtaacattgcgggctggctgctgggcaatccggaatgcgatccgctgctgccggttcgtagctggagctatattgtggaaaccccgaacagcgaaaacggcatttgctatccgggcgatttcatcgattatgaagaactgcgtgaacagctgtctagcgtgagcagctttgaacgctttgaaatcttcccgaaagaaagcagctggccgaaccataacaccaacggcgtgaccgcggcgtgtagccatgaaggcaaaagcagcttttatcgtaacctgctgtggctgaccgaaaaagaaggcagctatccgaaactgaaaaacagctacgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgtcgttttaccccggaaattgcggaacgtccgaaagtgcgtgatcaggcgggtcgtatgaactattattggaccctgctgaaaccgggcgataccattatttttgaagcgaacggcaacctgattgcgccgatgtatgcgtttgccctgagccgtggctttggcagcggcattattaccagcaacgcgagcatgcatgaactcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 64 - DNA, 5716 bp, artificial sequence
  chemically synthesized
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgaaaggcatcgcgccgctgcaactgggcaaatgtaacattgcgggctggctgctgggcaatccggaatgcgatccgctgctgccggttcgtagctggagctatattgtggaaaccccgaacagcgaaaacggcatttgctatccgggcgatttcatcgattatgaagaactgcgtgaacagctgtctagcgtgagcagctttgaacgctttgaaatcttcccgaaagaaagcagctggccgaaccataacaccaacggcgtgaccgcggcgtgtagccatgaaggcaaaagcagcttttatcgtaacctgctgtggctgaccgaaaaagaaggcagctatccgaaactgaaaaacagctacgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgtcgttttaccccggaaattgcggaacgtccgaaagtgcgtgatcaggcgggtcgtatgaactattattggaccctgctgaaaccgggcgataccattatttttgaagcgaacggcaacctgattgcgccgatgtatgcgtttgccctgagccgtggctttggcagcggcattattaccagcaacgcgagcatgcatgaactcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 65 - DNA, 5698 bp, artificial sequence
  chemically synthesized
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgaaaggcatcgcgccgctgcaactgggcaaatgtaacattgcgggctggctgctgggcaatccggaatgcgatccgctgctgccggttcgtagctggagctatattgtggaaaccccgaacagcgaaaacggcatttgctatccgggcgatttcatcgattatgaagaactgcgtgaacagctgtctagcgtgagcagctttgaacgctttgaaatcttcccgaaagaaagcagctggccgaaccataacaccaacggcgtgaccgcggcgtgtagccatgaaggcaaaagcagcttttatcgtaacctgctgtggctgaccgaaaaagaaggcagctatccgaaactgaaaaacagctacgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgtcgttttaccccggaaattgcggaacgtccgaaagtgcgtgatcaggcgggtcgtatgaactattattggaccctgctgaaaccgggcgataccattatttttgaagcgaacggcaacctgattgcgccgatgtatgcgtttgccctgagccgtggctttggcagcggcattattaccagcctcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 66 - DNA, 5689 bp, Artificial Sequence
  Plasmid pET42T_HA1_PR8_57_270
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatggcgccgctgcaactgggcaaatgtaacattgcgggctggctgctgggcaatccggaatgcgatccgctgctgccggttcgtagctggagctatattgtggaaaccccgaacagcgaaaacggcatttgctatccgggcgatttcatcgattatgaagaactgcgtgaacagctgtctagcgtgagcagctttgaacgctttgaaatcttcccgaaagaaagcagctggccgaaccataacaccaacggcgtgaccgcggcgtgtagccatgaaggcaaaagcagcttttatcgtaacctgctgtggctgaccgaaaaagaaggcagctatccgaaactgaaaaacagctacgtgaacaaaaaaggcaaagaagtgctggtgctgtggggcattcatcatccgccgaacagcaaagaacagcagaacctgtatcagaacgaaaacgcgtatgtgagcgtggtgaccagcaactataaccgtcgttttaccccggaaattgcggaacgtccgaaagtgcgtgatcaggcgggtcgtatgaactattattggaccctgctgaaaccgggcgataccattatttttgaagcgaacggcaacctgattgcgccgatgtatgcgtttgccctgagccgtggctttggcagcggcattattaccagcctcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 67 - protein, 288 aa, Artificial Sequence
  Protein gdHA_PR8_42_310
MLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRLEHHHHHHGGC

SEQ ID NO: 68 - protein, 284 aa, Artificial Sequence
  Protein gdHA_PR8_46_310
MSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRLEHHHHHHGGC

SEQ ID NO: 69 - protein, 238 aa, Artificial Sequence
  Protein gdHA_PR8_57_276
MAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHELEHHHHHHGGC

SEQ ID NO: 70 - protein, 241 aa, Artificial Sequence
  Protein gdHA_PR8_54a_276
MKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHELEHHHHHHGGC

SEQ ID NO: 71 - protein, 235 aa, Artificial Sequence
  Protein gdHA_PR8_54a_270
MKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSLEHHHHHHGGC

SEQ ID NO: 72 - protein, 232 aa, Artificial Sequence
  Protein gdHA_PR8_57_270
MAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSLEHHHHHHGGC

SEQ ID NO: 73 - protein, 509 aa, Influenza A virus
DTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGV

SEQ ID NO: 74 - protein, 503 aa, Influenza A virus
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGV

SEQ ID NO: 75 - protein, 329 aa, Influenza A virus
QDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTR

SEQ ID NO: 76 - protein, 176 aa, Influenza A virus
GLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGV

SEQ ID NO: 77 - DNA, 836 bp, Influenza A virus
tatgctgctggaagataaacataatggcaaactgtgtaaactgcgtggtgttgcaccgctgcatctgggtaaatgtaatattgccggttggattctgggtaatccggaatgtgaaagcctgagcaccgcaagcagctggtcttatattgttgaaaccccgagcagcgataatggcacctgttatccgggtgattttattgattatgaagaactgcgcgaacagctgagcagcgttagcagctttgaacgctttgaaatttttccgaaaaccagcagctggccgaatcatgatagcaataaaggtgttaccgcagcatgtccgcatgccggtgcaaaaagcttttacaaaaatctgatttggctggtgaaaaaaggtaatagctatccgaaactgagcaaaagctatatcaatgataaaggcaaagaagttctggttctttggggtattcatcatccgagcaccagcgcagatcagcagagcctgtatcagaatgcagatacctatgtttttgttggtagcagccgctatagcaaaaagtttaaaccggaaattgccattcgtccgaaagttcgtgatcaagagggtcgcatgaactattattggaccctggttgaaccgggtgacaaaattacctttgaagccaccggcaatctggttgttccgcgttatgcatttgcaatggaacgtaatgcaggtagcggcattatcattagcgatacaccggtgcatgattgtaataccacctgtcagaccccgaaaggtgcaattaataccagcctgccgtttcagaatattcatccgattaccattggtaaatgcccgaaatatgtgaaac

SEQ ID NO: 78 - DNA, 833 bp, Influenza A virus
tatgctgctggaaaatagccataatggtaaactgtgtctgctgaaaggtattgcaccgctgcagctgggtaattgtagcgttgcaggttggattctgggtaatccggaatgtgaactgctgattagcaaagaaagctggtcctatattgtggaaaaaccgaatccggaaaatggcacctgttatccgggtcattttgccgattatgaagaactgcgtgaacagctgagcagcgttagcagctttgaacgctttgaaatttttccgaaagaaagcagctggccgaatcataccgttaccggtgttagcgcaagctgttctcataatggcgaaagcagcttttatcgtaatctgctgtggctgaccggtaaaaatggtctgtatccgaatctgagcaaaagctatgccaataataaagaaaaagaagtgctggttctttggggtgttcatcatccgccgaatattggtgatcagaaagccctgtatcacaccgaaaatgcctatgttagcgttgttagcagccattatagccgtaaatttacaccggaaattgccaaacgtccgaaagttcgtgatcaggaaggtcgcattaattattattggaccctgctggaaccgggtgataccattatttttgaagccaatggcaatctgattgcaccgcgttatgcatttgcactgagccgtggttttggtagcggtattattaatagcaatgcaccgatggataaatgtgatgccaaatgtcagacaccgcagggtgcaattaatagcagcctgccgtttcagaatgttcatccggttaccattggtgaatgtccgaaatatgtgcgcc

SEQ ID NO: 79 - DNA, 812 bp, Influenza A virus
tatgctggttcagagcagcagcaccggtgaaatttgtgattctccgcatcagattctggatggtgaaaattgcaccctgattgatgcactgctgggtgatccgcagtgtgatggctttcagaataaaaaatgggacctgtttgtggaacgtagcaaagcctatagcaattgctatccgtatgatgttccggattatgcaagcctgcgtagcctggttgcaagcagcggcaccctggaatttaataatgaaagctttaattggaccggtgttacccagaatggcaccagcagcagctgtattcgtggtagcaataatagcttttttagccgtctgaattggctgacccatctgaaattcaaatatccggcactgaatgttaccatgccgaataatgaaaaatttgataaactgtatatttggggtgttcatcatccgggtacagataatgatcagatttttccgtatgcacaggcaagcggtcgtattaccgttagcaccaaacgtagccagcagaccgttattccgaatattggtagccgtccgcgtgttcgtaatattccgagccgcattagcatttattggaccattgtgaaaccgggtgatattctgctgattaatagcaccggtaatctgattgcaccgcgtggctattttaaaattcgcagcggcaaaagcagcattatgcgttctgatgcaccgattggtaaatgtaatagcgaatgcattaccccgaatggtagcattccgaatgataaaccgtttcagaatgtgaatcgcattacctatggtgcatgtccgcgttatgtgaaac

SEQ ID NO: 80 - DNA, 887 bp, Influenza A virus
tatgctgaccaccaccccgaccaaaagctatttcgccaatctgaaaggcaccaaaacccgtggtaaactgtgtccggattgtctgaattgtaccgatctggatgttgcactgggtcgtccgatgtgtgttggcaccaccccgagcgccaaagcaagcattctgcatgaagttcgtccggttaccagcggttgttttccgattatgcatgatcgtaccaaaattcgtcagctggcaaatctgctgcgtggctatgaaaacattcgtctgagcacccagaatgttattgatgcagaaaaagcaccgggtggtccgtatcgtctgggcaccagcggtagctgtccgaatgcaaccagcaaaagcggtttttttgcaaccatggcatgggcagttccgaaagataataataaaaatgccaccaatccgctgaccgttgaagttccgtatatttgcaccgaaggcgaagatcagattaccgtttggggttttcattccgatgataaaacccagatgaaaaatctgtatggcgatagcaatccgcagaaattcaccagcagcgcaaatggtgttaccacccattatgttagccagattggtggttttccggatcagaccgaagatggtggtctgccgcagagcggtcgtattgttgtggattacatgatgcagaaaccgggtaaaaccggcaccattgtttatcagcgtggtgttctgctgccgcagaaagtttggtgtgcaagcggtcgtagcaaagttattaaaggtagcctgccgctgattggtgaagcagattgcctgcatgaaaaatatggtggcctgaataaaagcaaaccgtattataccggtgaacatgcaaaagccattggtaattgtccgatttgggttaaac

SEQ ID NO: 81 - DNA, 833 bp, Influenza A virus
tatgattctggaaaaaaaacataatggcaaactgtgtgatctggatggtgttaaaccgctgattctgcgtgattgtagcgttgcaggttggctgctgggtaatccgatgtgtgatgaatttattaatgtgccggaatggtcctatattgtggaaaaagccaatccggttaatgatctgtgttatccgggtgattttaatgattatgaagaactgaaacatctgctgagccgcattaatcattttgaaaaaattcagattattccgaaaagcagctggtctagccatgaagcaagcctgggtgttagcagcgcatgtccgtatcagggtaaaagcagcttttttcgcaatgttgtgtggctgattaaaaaaaatagcacctatccgaccattaaacgcagctataataataccaatcaagaggatctgctggttctgtggggtattcatcatccgaatgatgcagcagaacagaccaaactgtatcagaatccgaccacctatattagcgttggcaccagcaccctgaatcagcgtctggttccgcgtattgcaacccgtagcaaagttaatggtcagagcggtcgcatggaatttttttggaccattctgaaaccgaatgatgccattaattttgaaagcaatggcaattttattgcaccggaatatgcctataaaattgtgaaaaaaggcgatagcaccattatgaaaagcgaactggaatatggcaattgcaataccaaatgtcagaccccgatgggtgcaattaatagcagcatgccgtttcataacattcatccgctgaccattggtgaatgtccgaaatatgtgaaac

SEQ ID NO: 82 - DNA, 833 bp, Influenza A virus
tatgattctggaaaaaacccataatggcaaactgtgtgatctggatggtgttaaaccgctgattctgcgtgattgtagcgttgcaggttggctgctgggtaatccgatgtgtgatgaatttattaatgtgccggaatggtcctatattgtggaaaaagccaatccgaccaatgatctgtgttatccgggtagctttaatgattatgaagaactgaaacatctgctgagccgcattaatcattttgaaaaaattcagattattccgaaaagcagctggtctgatcatgaagcaagcagcggtgttagcagcgcatgtccgtatctgggtagcccgagcttttttcgtaatgtggtgtggctgattaaaaaaaatagcacctatccgaccattaaaaaaagctataataataccaatcaagaggatctgctggttctgtggggtattcatcatccgaatgatgcagcagaacagacccgtctgtatcagaatccgaccacctatattagcattggcaccagcaccctgaatcagcgtctggttccgaaaattgcaacccgtagcaaagttaatggtcagagcggtcgcatggaatttttttggaccattctgaaaccgaatgatgccattaattttgaaagcaatggcaattttattgcaccggaatatgcctataaaattgtgaaaaaaggcgatagcgccattatgaaaagcgaactggaatatggcaattgcaataccaaatgtcagaccccgatgggtgcaattaatagcagcatgccgtttcataacattcatccgctgaccattggtgaatgtccgaaatatgtgaaac

SEQ ID NO: 83 - DNA, 5923 bp, artificial sequence
  chemically synthesized
gtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatcgatatcgaattctaaggaggaaaaaaaaatgctgctggaagataaacataatggcaaactgtgtaaactgcgtggtgttgcaccgctgcatctgggtaaatgtaatattgcaggttggattctgggtaatccggaatgtgaaagcctgagcaccgcaagcagctggtcatatattgttgaaaccccgagcagcgataatggcacctgttatccgggtgattttattgattatgaagaactgcgcgaacagctgagcagcgttagcagctttgaacgttttgaaatttttccgaaaaccagcagctggccgaatcatgatagcaataaaggtgttaccgcagcatgtccgcatgccggtgcaaaaagtttttataaaaatctgatttggctggtgaaaaaaggcaatagctatccgaaactgagcaaaagctatattaatgataaaggcaaagaagtgctggtgctgtggggtattcatcatccgagcaccagcgcagatcagcagagcctgtatcagaatgcagatgcatatgtttttgttggtagcagccgctatagcaaaaaatttaaaccggaaattgccattcgtccgaaagttcgtgatcgtgaaggtcgtatgaattattattggaccctggttgaaccgggtgataaaattacctttgaagcaaccggcaatctggttgttccgcgttatgcatttgcaatggaacgtaatgcaggtagcggcattattattagcgatacaccggttcatgattgcaataccacctgtcagaccccgaaaggtgcaattaataccagcctgccgtttcagaatattcatccgattaccattggcaaatgcccgaaatatgtgaaaggtggttgcggctaataataaaagcttctgcagctgctcgagcaccaccaccaccaccacggtggttgctaataataattgattaatacctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggt

SEQ ID NO: 84 - protein, 278 aa, Influenza A virus
MLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVK

SEQ ID NO: 85 - protein, 277 aa, Influenza A virus
MLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVR

SEQ ID NO: 86 - protein, 270 aa, Influenza A virus
MLVQSSSTGEICDSPHQILDGENCTLIDALLGDPQCDGFQNKKWDLFVERSKAYSNCYPYDVPDYASLRSLVASSGTLEFNNESFNWTGVTQNGTSSSCIRGSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVK

SEQ ID NO: 87 - protein, 295 aa, Influenza B virus
MLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVK

SEQ ID NO: 88 - protein, 277 aa, Influenza A virus
MILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVK

SEQ ID NO: 89 - protein, 277 aa, Influenza A virus
MILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVK

SEQ ID NO: 90 - protein, 278 aa, Influenza A virus
MLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDREGRMNYYWTLVEPGDKITFEATGNLVVPRYAFAMERNAGSGIIISDTPVHDCNTTCQTPKGAINTSLPFQNIHPITIGKCPKYVK

SEQ ID NO: 91 - protein, 11 aa, artificial sequence
  chemically synthesized
LEHHHHHHGGC

SEQ ID NO: 92 - protein, 122 aa, Bacteriophage phi 42
ALGDTLTITLGGSGGTAKVLRKINQDGYTSEYYLPETSSSFRAKVRHTKESVKPNQVQYERHNVEFTETVYASGSTPEFVRQAYVVIRHKVGDVSATVSDLGEALSFYLNEALYGKLIGWES

SEQ ID NO: 93 - protein, 122 aa, Bacteriophage phiCB5
ALGDTLTITLGGSGGTAKVLKKINQDGYTSEYYLPETSSSFRAKVRHTKESVKPNQVQYERHNVEFTETVYASGSTPEFVRQAYVVIRHKVGDVSATVSDLGEALSFYLNEALYGKLIGWES

SEQ ID NO: 94 - protein, 122 aa, Bacteriophage phiCb5
ALGDTLTITLGGSGGTAKVLKKINQDGYTSEYYLPETSSSFRAKVRHTKESVKPNQVQYERHNVEFTETVYASCCTPEFVRQAYVVIRHKVGDVSATVSDLGEALSFYLNEALYGKLIGWES

SEQ ID NO: 95 - protein, 30 aa, bacteriophage T4
GSGYIPEAPRDGQAYVRKDGEWVLLSTFLG

SEQ ID NO: 96 - DNA, 10 bp, artificial sequence
  chemically synthesized
gacgatcgtc

SEQ ID NO: 97 - DNA, 22 bp, artificial sequence
  chemically synthesized
gggggggacgatcgtcgggggg

SEQ ID NO: 98 - DNA, 24 bp, artificial sequecnce
  chemically synthesized
ggggggggacgatcgtcggggggg

SEQ ID NO: 99 - DNA, 26 bp, artificial sequence
  chemically synthesized
gggggggggacgatcgtcgggggggg

SEQ ID NO: 100 - DNA, 28 bp, artificial sequence
  chemically synthesized
ggggggggggacgatcgtcggggggggg

SEQ ID NO: 101 - DNA, 30 bp, artificial sequence
  chemically synthesized
gggggggggggacgatcgtcgggggggggg

(19) United States
(12) Patent Application Publication
(10) Pub. No.: US 2012/0263743 A1
(43) Pub. Date: Oct. 18, 2012

(54) INFLUENZA HEMAGGLUTININ COMPOSITIONS AND USES THEREOF

(75) Inventors: Martin Bachmann, Ramismuhle (CH); Andrea Jegerlehner, Zurich (CH); Philippe Saudan, Pfungen (CH)

(73) Assignee: Cytos Biotechnology AG, zURICH-sCHLIEREN (CH)

(21) Appl. No.: 13/318,099

(22) PCT Filed: Apr. 30, 2010
(86) PCT No.: PCT/EP2010/055944

§ 371 Date: Jun. 21, 2012

(30) Foreign Application Priority Data
Apr. 30, 2009 (EP) 09159262.6

(51) Int. Cl. A61K 39/145; A61P 31/16; A61P 37/04; C07K 14/11

(52) U.S. Cl. 424/186.1; 530/322; 530/395

(57) ABSTRACT

The present invention is in the fields of medicine, public health, immunology, molecular biology and virology. The invention provides compositions, vaccine compositions and pharmaceutical compositions for the treatment, amelioration and/or prevention of influenza. The compositions, vaccine compositions and pharmaceutical compositions of the invention comprise a virus-like particle of an RNA bacteriophage and at least one antigen, wherein said at least one antigen is an ectodomain of an influenza virus hemagglutinin protein or a fragment of said ectodomain of an influenza virus hemagglutinin protein. When administered to an animal, preferably to a human, said compositions, vaccine compositions and pharmaceutical compositions efficiently induce immune responses, in particular antibody responses, wherein typically and preferably said antibody responses are directed against influenza virus. Thus, the invention further provides methods of treating, ameliorating and/or preventing influenza virus infection.

The present invention is in the fields of medicine, public health, immunology, molecular biology and virology. The invention provides compositions, vaccine compositions and pharmaceutical compositions for the treatment, amelioration and/or prevention of influenza. The compositions, vaccine compositions and pharmaceutical compositions of the invention comprise a virus-like particle of an RNA bacteriophage and at least one antigen, wherein said at least one antigen is an ectodomain of an influenza virus hemagglutinin protein or a fragment of said ectodomain of an influenza virus hemagglutinin protein. When administered to an animal, preferably to a human, said compositions, vaccine compositions and pharmaceutical compositions efficiently induce immune responses, in particular antibody responses, wherein typically and preferably said antibody responses are directed against influenza virus. Thus, the invention further provides methods of treating, ameliorating and/or preventing influenza virus infection.

### RELATED ART

The emergence of high pathogenicity avain influenza viruses in domestic poultry and the increasing number of cases of transmission of avian influenza viruses or porcine viruses of different subtypes to humans and the subsequent direct transmission of those viruses within the human population are significant threat to public health because of the potential for pandemic spread of these viruses (Subbarao et al. 2007, Nature reviews 7:267-278).

There are three types of influenza viruses, influenza A, B and C. Influenza B virus almost exclusively infects humans and contains only one type of main surface glycoproteins, hemagglutinin (HA) and neuraminidase (NA).

Influenza A viruses are classified into different subtypes on the basis of genetic and antigenic differences in their main surface glycoproteins, hemagglutinin (HA) and neuraminidase (NA) (Wright et al. 2001, Fields Virology 4th edn.; Eds Knipe D. M. & Howley, P. M. 1533-1579). There are at least 16 different HA antigens known. These subtypes are named from H1 through H16.

The HA protein mediates the attachment of the virus to the host cell and viral-cell membrane fusion during penetration of the virus into the cytosol of the cell. The influenza virus genome consists of eight single-stranded negative-sense RNA segments of which the fourth largest segment encodes the HA protein.

Influenza HA is a homotrimeric integral membrane glycoprotein which is present on the surface of the virion and on infected cells. The HA protein is anchored in the membrane through a transmembrane region which is spanning sequences of each of the three monomers. The main protective efficacy of influenza vaccines is attributed to anti-hemagglutinin antibodies which inhibit the attachment and hence infection of the cells (Virelizier J. L. 1975 J. Immunol. 115:434-439). Inhibition of virus attachment protects individuals against infection or serious illness. The degree of protection correlates with the magnitude of anti-HA titers. The HA glycoprotein is synthesized as a HA0 precursor that is post-translationally cleaved into HA1 and HA2 subunits. This cleavage occurs N-terminaly of the fusion peptide and is essential for fusion to occur (Steinhauer D. A. 1999 Virology 258:1-20). The fusion process requires that HA forms homotrimers (Danieli et al. 1996 J. Cell Biol. 133:559-569). Influenza viruses are described by a nomenclature which includes the type, geographic origin, strain number, year of isolation and HA and NA subtype, for example, A/California/04/09) (H1N1). There are at least 16 HA subtypes (H1-H16) and 9 NA (N-1-N9) subtypes known. (Murphy and Webster, “Orthomyxoviruses”, in Virology, ed. Fields, B. N., Knipe, D. M., Chanock, R. M., 1091-1152 (Raven Press, New York 1990)). Six of the 16 HA subtypes, being H1, H2, H3, H5, H7 and H9 have already been identified in influenza A viruses that infect humans (Cox et al., 2003 Scandanavian J. of Immun. 59:1-15).

Antibodies directed against HA can neutralize influenza infection and are the basis for natural immunity against influenza (Clements, “influenza Vaccines”, in Vaccines: New Approaches to Immunological Problems, ed. Ronald W. Ellis, pp. 129-150 (Butterworth-Heinemann, Stoneham, Mass. 1992). Antigenic variation within the HA molecule is responsible for frequent outbreaks of influenza and for limited control of infection by vaccination. The HA part of influenza virus is the target of the protective immune response and can vary as a result of antigenic drift and antigenic shift.

Antigenic drift refers to small, gradual changes that occur through point mutations in the two genes that contain the genetic material to produce the main surface proteins, hemagglutinin, and neuraminidase. These point mutations occur unpredictably and result in minor changes to these surface proteins. Antigenic drift produces new virus strains that may not be recognized by antibodies to earlier influenza strains. This is one of the main reasons why people can become infected with influenza viruses more than once and why global surveillance is critical in order to monitor the evolution of human influenza virus stains for selection of those strains which should be included in the annual production of influenza vaccine. In most years, one or two of the three virus strains in the influenza vaccine are updated to keep up with the changes in the circulating influenza viruses. For this reason, people who want to be immunized against influenza need to be vaccinated every year (Center for Disease control and Prevention Subbarao et al. 2007 Nature reviews 7:267-278). Antigenic shift is a phenomenon observed for influenza A virus. It refers to an abrupt, major change which is resulting in a novel influenza A virus subtype in humans that was not currently circulating among people. Antigenic shift can occur either through direct animal-to-human transmission or through mixing of human influenza A and animal influenza A virus genes to create a new human influenza A subtype virus through a process called genetic reassortment. A global influenza pandemic (worldwide spread) may occur if three conditions are met: (i) a new subtype of influenza A virus is introduced into the human population; (ii)

the virus causes serious illness in humans; (iii) the virus can spread easily from person to person in a sustained manner.

The majority of marketed influenza vaccines is produced in embryonated chicken eggs. The use of eggs to grow the annual flu vaccine has several well-known disadvantages, particularly the inability to rapidly produce vaccines in response to epidemics or pandemics conditions. Approaches which are based on recombinant expression of the antigen have been investigated as alternatives for new influenza vaccines. In theses vaccines the protein antigens are produced in prokaryotic and eukaryotic expression systems such as E. coli, yeast, insect cells, and mammalian cells. The development of recombinant subunit vaccines for influenza is an attractive option because the need to grow viruses is eliminated.

Two major problems have hampered the development of recombinant influenza proteins. On one hand the low expression levels and on the other hand the difficulty to express proteins with the native conformation in prokaryotic cells. For example, HA, the primary component for influenza vaccines, has proven to be difficult to express recombinantly. Expression in Pichia of a membrane anchorless HA molecule has been reported (Saelens et al., 1999 Eur. J. Biochem. 260:166-175). In another study, Mc Ewen et al. (1992 Vaccine; 10:405-411) have shown that a synthetic peptide containing an 18 amino acid residue epitope of the HA molecule of the H3 subtype of influenza, cloned into the flagellin gene of Salmonella, is able to induce local IgA in the lungs, and to provide partial protection against influenza challenge in a mouse model. Similarly, Jeon et al. (2002 Viral Immunology 15:165-176) reported that mice which were immunized with the protein fragment HA91-261 induced significant protection against viral challenge based on hemagglutination assay in lung homogenates. Song et al. (2008 PLoS one 3:e2257) have generated vaccines, wherein the globular head domain of HA antigen is fused with the potent TLR5 ligand flagellin.

### SUMMARY OF THE INVENTION

In its main aspect the present invention relates to compositions comprising: (a) a virus-like particle (VLP) with at least one first attachment site, wherein preferably said virus-like particle is a virus-like particle of an RNA bacteriophage; and (b) at least one antigen with at least one second attachment site, wherein said at least one antigen is an ectodomain of an influenza virus hemagglutinin protein or a fragment of said ectodomain of an influenza virus hemagglutinin protein, wherein said fragment of said ectodomain of an influenza virus hemagglutinin protein comprises at least 80 contiguous amino acids of said ectodomain of an influenza virus hemagglutinin protein; and wherein (a) and (b) are linked through said at least one first and said at least one second attachment site. We have, now, surprisingly found that the inventive compositions are capable of inducing immune responses, in particular antibody responses, leading to high antibody titers which protect against a lethal challenge with an influenza virus in an animal model for influenza.

### DETAILED DESCRIPTION OF THE INVENTION

Adjuvant:

The term “adjuvant” as used herein refers to non-specific stimulators of the immune response or substances that allow generation of a depot in the host which, when combined with the vaccine composition or pharmaceutical composition of the invention, provide for a more enhanced immune response than said vaccine composition or pharmaceutical composition alone. Adjuvant includes (a) mineral gels, preferably aluminum hydroxide; (b) surface active substances, including lysolecithin, pluronic polyols, polyanions, peptides, oil emulsions, keyhole limpet hemocyanins, or dinitrophenol; and (c) human adjuvants, preferably BCG (bacille Calmette Guerin) and Corynebacterium parvum. Adjuvant further includes complete and incomplete Freund's adjuvant, modified muramyldipeptide, monophosphoryl lipid immunomodulator, AdjuVax 100a, QS-21, QS-18, CRL1005, MF-59, OM-174, OM-197, OM-294, and virosomal adjuvant technology. Preferred adjuvant is aluminum containing adjuvant, preferably aluminum salt, most preferably aluminum hydroxide (Alum). The term adjuvant also encompasses mixtures of these substances. VLP have been generally described as an adjuvant. However, the term “adjuvant”, as used within the context of this application, refers to an adjuvant not being the VLP comprised by the inventive compositions, vaccine compositions and/or pharmaceutical compositions. Rather, the term adjuvant relates to an additional, distinct component of said compositions, vaccine compositions and/or pharmaceutical compositions.

Antigen:

As used herein, the term “antigen” refers to a molecule capable of being bound by an antibody or a T-cell receptor (TCR) if presented by MHC molecules. The term “antigen”, as used herein, also refers to T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and/or is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. If not indicated otherwise, the term “antigen” as used herein does not refer to the virus-like particle contained in the inventive compositions, vaccine compositions and/or pharmaceutical compositions.

“Corresponding” Amino Acid Positions (H3 Numbering):

The amino acid sequences of the HA1 and of the HA2 subunits of influenza virus hemagglutinin proteins are highly variable. Therefore, the amino acid positions of these subunits are typically not addressed directly but they are mapped to amino acid positions of the amino acid sequences of the HA1 and of HA2 subunit of a reference strain of influenza virus, preferably by way of structural alignment. The reference strain which is generally used in the art and which is also used herein is the human influenza A virus H3 1968 (Wilson et al. 1981, Nature 289:366-373). Accordingly, amino acid positions of hemagglutinin HA1 subunits are mapped to the HA1 subunit of human influenza A virus H3 1968 (SEQ ID NO:75), and amino acid positions of hemagglutinin HA2 subunits are mapped to the HA2 subunit of human influenza A virus H3 1968 (SEQ ID NO:76), preferably by structural alignment. The resulting numbering system of the amino acid positions is therefore often referred to as “H3 numbering”. Typically and preferably the structural alignment is performed based on crystal structure data. Crystal structure data are available for subtypes H1 (Gamblin et al. 2004 Science 303:1838-1842, and references cited therein), H3 (Wilson et al. 1981, Nature 289:366-373), H5 (Stevens et al. 2006, Science 312:404-410). Structural information for HA subtypes for which no crystal structure is available can be obtained by structure model building based on the amino acid sequence. For the purpose of the invention structure model building is preferably performed by the software SWISS-MODEL. Tools and algorithms to generate alignments which are based on structural data are readily available to the artisan (e.g. Weis W I et al. 1990, Refinement of the influenza virus hemagglutinin by simulated annealing. J Mol. Biol. 1990 Apr. 20; 212(4):737-61.). Typically and preferably, the mapping of the amino acid positions of a given HA1 or HA2 subunit of influenza A subtypes H1, H2, H3, H5 and H9 is based on the alignment which is provided Stevens et al. 2004 (Science 303:1866-1870, supplemental online materials, Figure S1). The Structure of influenza B virus hemagglutinin is known from Wang et al. 2008 (J. Virol., p. 3011-3020). Typically and preferably, the H3 mapping of the amino acid positions of a given influenza B virus hemagglutinin HA1 subunit is based on the alignment which is provided by Tung et al. 2004 (J Gen Virol. 85:3249-59). A given amino acid sequence is referred to as corresponding to certain amino acid positions on a reference amino acid sequence, when said given amino acid sequence can be mapped, i.e. structurally aligned, to a contiguous section of said reference amino acid sequence, wherein said contiguous section is defined by said amino acid positions. Typically and preferably, a given amino acid sequence which is corresponding to certain amino acid positions on a reference amino acid sequence does not comprise any flanking sequences which can not be mapped to the reference amino acid sequence. Thus, the terms “an amino acid sequence corresponding to amino acid position 11 to amino acid position 328 of SEQ ID NO:75”, “an amino acid sequence corresponding to amino acid position 11 to amino acid position 329 of SEQ ID NO:75”, “an amino acid sequence corresponding to amino acid position 1 to 176 of SEQ ID NO:76”, or the like such as “an amino acid sequence corresponding to an amino acid sequence consisting of position 115 to position 261 of SEQ ID NO:75” refer to an amino acid sequence which can be mapped, i.e. structurally aligned, to that contiguous section of the reference amino acid sequence which is defined by the position numbers.

Ectodomain of an Influenza Virus Hemagglutinin Protein (HA Ectodomain):

As used herein, the term “ectodomain of an influenza virus hemagglutinin protein” (HA ectodomain) refers to (i) a protein, wherein said protein is composed of (a) the HA1 subunit comprising or preferably consisting of amino acid position 11 to amino acid position 328 of SEQ ID NO:75 and (b) the HA2 subunit consisting of position 1 to 176 of SEQ ID NO:76, and (ii) to any protein having an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% therewith, wherein further preferably said HA ectodomain is a naturally occurring HA ectodomain. The term “ectodomain of an influenza virus hemagglutinin protein” preferably refers to a protein selected from the group consisting of: (i) a protein composed of (a) the HA1 subunit consisting of amino acid position 11 to amino acid position 329 of SEQ ID NO:75 and (b) the HA2 subunit consisting of position 1 to 176 of SEQ ID NO:76; (ii) a protein composed of (a) the HA1 subunit consisting of amino acid position 11 to amino acid position 328 of SEQ ID NO:75 and (b) the HA2 subunit consisting of position 1 to 176 of SEQ ID NO:76; (iii) a protein composed of (a) a HA1 subunit of a naturally occurring influenza virus hemagglutinin protein, wherein said HA1 subunit of said naturally occurring influenza virus hemagglutinin protein consists of an amino acid sequence corresponding to amino acid position 11 to amino acid position 329 of SEQ ID NO:75 and (b) a HA2 subunit of a naturally occurring influenza virus hemagglutinin protein, wherein said HA2 subunit of said naturally occurring influenza virus hemagglutinin protein consists of an amino acid sequence corresponding to amino acid position 1 to 176 of SEQ ID NO:76; (iv) a protein composed of (a) a HA1 subunit of a naturally occurring influenza virus hemagglutinin protein, wherein said HA1 subunit of said naturally occurring influenza virus hemagglutinin protein consists of an amino acid sequence corresponding to amino acid position 11 to amino acid position 328 of SEQ ID NO:75 and (b) a HA2 subunit of a naturally occurring influenza virus hemagglutinin protein, wherein said HA2 subunit of said naturally occurring influenza virus hemagglutinin protein consists of an amino acid sequence corresponding to amino acid position 1 to 176 of SEQ ID NO:76; and (v) a protein having an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with any one of the proteins defined in (i), (ii), (iii), or (iv), wherein further preferably said HA ectodomain is a naturally occurring HA ectodomain. In a HA ectodomain according to the invention said HA1 subunit (a) is typically and preferably bound to said HA2 subunit (b) by way of at least one, preferably by one or two, covalent bond(s), wherein preferably said covalent bond(s) are selected from the group consisting of peptide bond and disulfide bond. Very preferably, said HA1 subunit (a) is bound to said HA2 subunit (b) by way of at least one, preferably by one or two, covalent bond(s), wherein at least one of said covalent bonds is a disulfide bond. Very preferably, said HA1 subunit (a) is genetically fused to the N-terminus of said HA2 subunit (b), wherein said HA1 subunit (a) is further bound to said HA2 subunit (b) by at least one, preferably one, disulfide bond. It is to be understood that in certain embodiments of the invention the peptide bond between said HA1 and said HA2 subunit may be cleaved during the maturation of the fusion product, wherein said disulfide bond remains intact. Thus, said HA1 subunit (a) is preferably bound to said HA2 subunit (b) by way of exactly one covalent bond, wherein said covalent bond is a disulfide bond. However, HA ectodomains being fusion products of HA1 and HA2, wherein the peptide bond between the HA1 and the HA2 subunit remains intact are also encompassed by the invention. Thus, in a further preferred HA ectodomain according to the invention said HA1 subunit (a) is genetically fused to the N-terminus of said HA2 subunit (b), wherein said HA1 subunit (a) is bound to said HA2 subunit (b) by way of one first covalent bond and by at least one, preferably one, second covalent bond, wherein said first covalent bond is a peptide bond and wherein said at least one second covalent bond is a disulfide bond.

“Naturally Occurring”:

The term “naturally occurring”, with respect to an influenza virus or to an influenza virus strain, refers to an influenza virus or to an influenza virus strain which is present in a natural host population, preferably in the human population. Typically and preferably, a naturally occurring influenza virus or influenza virus strain is isolated from an infected individual of said population. With respect to an influenza virus hemagglutinin protein or with respect to a HA ectodomain, the term “naturally occurring” refers to an influenza virus hemagglutinin protein or to a HA ectodomain of a natural occurring influenza virus or of a naturally occurring influenza virus strain.

Fragment of Said Ectodomain of an Influenza Virus Hemagglutinin Protein:

As used herein, the term “fragment of said ectodomain of an influenza virus hemagglutinin protein” refers to a portion of influenza virus hemagglutinin protein and contains at least 80, or at least 100, or at least 150, or at least 180, or at least 190, or at least 200 or at least 210, or at least 220, or at least 230, or at least 250, or at least 270, or at last 290 or at least 310 or at least 320 consecutive amino acids of the ectodomain of an influenza virus hemagglutinin protein of influenza A or B virus, preferably of the HA1 subunit of the ectodomain of an influenza virus hemagglutinin protein. The term fragment of said ectodomain of an influenza virus hemagglutinin protein also includes portions of influenza virus hemagglutinin protein, wherein said fragment is derived by deletion of one or more amino acids at the N and/or C terminus of said ectodomain of an influenza virus hemagglutinin protein. The fragment of said ectodomain of an influenza virus hemagglutinin protein preferably comprises certain elements of its secondary structure. Such structural elements can readily be identified by the artisan based on the structural data which are available from the prior art. In a very preferred embodiment, said fragment of said ectodomain of an influenza virus hemagglutinin protein comprises at least one eight-stranded Jelly roll barrel and at least one α-helix of the influenza virus hemagglutinin protein. In a preferred embodiment said fragment of said ectodomain of an influenza virus hemagglutinin protein comprises, or preferably consists of, a receptor binding domain. In a further preferred embodiment said fragment of said ectodomain of an influenza virus hemagglutinin protein further comprises a vestigial esterase domain. Typically and preferably said fragment of said ectodomain of an influenza virus hemagglutinin protein comprises at least one and at most four pair(s) of cysteine residues which are capable of forming intramolecular disulfide bond(s). More preferably, said fragment of said ectodomain of an influenza virus hemagglutinin protein comprises two pairs of cysteine residues which are capable of forming intramolecular disulfide bonds. The fragment of said ectodomain of an influenza virus hemagglutinin protein is preferably obtained by recombinant expression in eukaryotic or prokaryotic expression systems, preferably in a prokaryotic expression system, most preferably in E. coli. Typically and preferably said fragment of said ectodomain of an influenza virus hemagglutinin protein, when covalently bound to a virus-like particle according to the invention, is capable of inducing hemagglutination of red blood cells, wherein said red blood cells are preferably derived from chicken, turkey, horse, or human. A fragment of said ectodomain of an influenza virus hemagglutinin protein which is bound to a virus-like particle according to the invention, is hereby considered as being capable of inducing hemagglutination of red blood cells when hemagglutination is observed at a concentration of 0.50 μg or less of the conjugate/1 μl of 1% red blood cells. The hemagglutination assay is hereby preferably performed as described in Example 35.

Position 54a of the HA1 Subunit of Said Ectodomain of an Influenza Virus Hemagglutinin Protein:

The naturally occurring amino acid sequence of an influenza virus A or B may have an insertion of a heterologous amino acid residue. For example, position “54a” refers to the insertion as described in FIG. 1 of Russell et al. 2004 (Virology 325:287-296). Thus, for the influenza A subtype H1, the amino acid at position 54a is Lysine.

Associated:

The terms “associated” or “association” as used herein refer to chemical and/or physical interactions, by which two molecules are joined together. Chemical interactions include covalent and non-covalent interactions. Preferred non-covalent interactions are ionic interactions, hydrophobic interactions or hydrogen bonds. Preferred covalent interactions are covalent bonds, most preferably ester, ether, phosphoester, amide, peptide, carbon-phosphorus bonds, carbon-sulfur bonds such as thioether, or imide bonds.

Attachment Site, First:

As used herein, “first attachment site” refers to an element which is naturally occurring with the VLP or which is artificially added to the VLP, and to which the second attachment site can be linked. The first attachment site preferably comprises or is a chemically reactive group, preferably an amino group, a carboxyl group, a sulfhydryl group, a hydroxyl group, a guanidinyl group, histidinyl group, or a combination thereof. Very preferably, the first attachment site comprises or is an amino group. The term first attachment site therefore also includes proteins, polypeptides, peptides, and preferably an amino acid residues. The term first attachment site further includes other reactive chemical residues including sugars, biotin, fluorescein, retinol, and digoxigenin. In a preferred embodiment the first attachments site is a chemically reactive group, preferably the amino group of an amino acid residue, most preferably the amino group of a lysine residue. In a further preferred embodiment the first attachment site is an amino group or a carboxyl group, preferably an amino group or a carboxyl group of an amino acid residue. The first attachment site is preferably located on the surface, and most preferably on the outer surface of the VLP. Further preferably, multiple first attachment sites are present on the surface, preferably on the outer surface of the VLP, typically and preferably in a repetitive configuration. In a preferred embodiment the first attachment site is associated with the VLP, through at least one covalent bond, preferably through at least one peptide bond. In a further preferred embodiment the first attachment site is naturally occurring with the VLP. In a very preferred embodiment said first attachment site is an amino group of an amino acid residue of a protein comprised by the VLP, wherein further preferably said first attachment site is an amino group of a lysine residue comprises by a protein of the VLP. In a further very preferred embodiment said first attachment site is an amino group of an amino acid residue of a coat protein comprised by the VLP, wherein further preferably said first attachment site is an amino group of a lysine residue comprises by a coat protein of the VLP. Alternatively, in a preferred embodiment the first attachment site is artificially added to the VLP.

Attachment Site, Second:

As used herein, “second attachment site” refers to an element which is naturally occurring with or which is artificially added to the antigen and to which the first attachment site can be linked. The second attachment site of the antigen preferably is a protein, a polypeptide, a peptide, an amino acid, a sugar, or a chemically reactive group such as an amino group, a carboxyl group, or a sulfhydryl group. In a preferred embodiment the second attachment site is a chemically reactive group, preferably a chemically reactive group of an amino acid. In a very preferred embodiment the second attachment site is a sulfhydryl group, preferably a sulfhydryl group of an amino acid, most preferably a sulfhydryl group of a cysteine residue. In a further preferred embodiment the second attachment site is an amino group or a carboxy group, preferably an amino group or a carboxy group of an amino acid residue. The term “antigen with at least one second attachment site” refers, therefore, to a construct comprising the antigen and at least one second attachment site. In one embodiment, the second attachment site is naturally occurring within the antigen. In another embodiment, the second attachment site is artificially added to the antigen, preferably through a linker. Thus, an antigen with at least one second attachment site, wherein said second attachment site is not naturally occurring within said antigen, typically and preferably further comprises a “linker”. In a preferred embodiment the second attachment site is associated with the antigen through at least one covalent bond, preferably through at least one peptide bond.

Linker:

A “linker”, as used herein, either associates the second attachment site with the antigen or comprises, essentially consists of, or consists of the second attachment site. Preferably, the “linker” comprises or alternatively consists of the second attachment site, wherein further preferably said second attachment is one amino acid residue, preferably a cysteine residue. A linker comprising at least one amino acid residue is also referred to as amino acid linker. In a very preferred embodiment, the linker is an amino acid linker, wherein preferably said amino acid linker consists exclusively of amino acid residues. Further preferred embodiments of a linker in accordance with this invention are molecules comprising a sulfhydryl group or a cysteine residue. Association of the linker with the antigen is preferably by way of at least one covalent bond, more preferably by way of at least one peptide bond. In the context of linkage of the VLP and the antigen by genetic fusion, a linker may be absent or preferably is an amino acid linker, more preferably an amino acid linker consisting exclusively of amino acid residues.

Ordered and Repetitive Antigen Array:

As used herein, the term “ordered and repetitive antigen array” refers to a repeating pattern of antigen. An ordered and repetitive antigen array is characterized by a typically and preferably high order of uniformity in the spatial arrangement of the antigen with respect to virus-like particle. In one embodiment of the invention, the repeating pattern is a geometric pattern. A preferred ordered and repetitive antigen array is formed by antigen which is coupled to a VLP of an RNA bacteriophage. An ordered and repetitive antigen array formed by antigen which is coupled to a VLP of an RNA bacteriophage, typically and preferably possess strictly repetitive paracrystalline orders of antigen, preferably with spacing of 1 to 30 nanometers, preferably 2 to 15 nanometers, even more preferably 2 to 10 nanometers, even again more preferably 2 to 8 nanometers, and further more preferably 1.6 to 7 nanometers.

Polypeptide:

The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

Sequence Identity (Amino Acid Sequences):

The percentage of sequence identity between two given amino acid sequences is determined using any standard algorithm, preferably by the algorithm implemented in the Bestfit program. Typically and preferably the default parameter settings of said algorithms, preferably of the Bestfit algorithms are applied. This method is applicable to the determination of the sequence identity between the amino acid sequences of any protein, polypeptide or a fragment thereof disclosed in the invention.

Coat Protein:

The term “coat protein” refers to a viral protein, preferably to a subunit of a natural capsid of a virus, preferably of an RNA bacteriophage, which is capable of being incorporated into a virus capsid or a VLP. The term coat protein encompasses naturally occurring coat protein as well as recombinantly expressed coat protein. Further encompassed are mutants and fragments of coat protein, wherein said mutants and fragments retains the capability of forming a VLP.

Virus-Like Particle (VLP):

as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering a host cell. Preferably, a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA bacteriophage. The terms “viral capsid” or “capsid”, refer to a macromolecular assembly composed of viral protein subunits, wherein preferably said viral protein subunits are coat proteins of said virus. Typically, there are 60, 120, 180, 240, 300, 360 and more than 360 viral protein subunits, preferably coat protein subunits. Typically and preferably, the interactions of these subunits lead to the formation of viral capsid with an inherent repetitive organization, wherein said structure is, typically, spherical or tubular. For example, the capsids of RNA bacteriophages have a spherical form of icosahedral symmetry. One feature of a virus-like particle is its highly ordered and repetitive arrangement of its subunits.

Virus-Like Particle of an RNA Bacteriophage:

As used herein, the term “virus-like particle of an RNA bacteriophage” refers to a virus-like particle comprising, or preferably consisting essentially of or consisting of coat proteins, mutants or fragments thereof, of an RNA bacteriophage. In addition, virus-like particle of an RNA bacteriophage resembling the structure of an RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. Also included are virus-like particles of RNA bacteriophages, in which the aforementioned gene or genes are still present but inactive, and, therefore, also leading to non-replicative and/or non-infectious virus-like particles of an RNA bacteriophage. Preferred VLPs derived from RNA bacteriophages exhibit icosahedral symmetry and consist of 180 subunits (monomers). Preferred methods to render a virus-like particle of an RNA bacteriophage non replicative and/or non-infectious is by physical, chemical inactivation, such as UV irradiation, formaldehyde treatment, typically and preferably by genetic manipulation.

Recombinant VLP:

The term “recombinant VLP”, as used herein, refers to a VLP that is obtained by a process which comprises at least one step of recombinant DNA technology. Typically and preferably a recombinant VLP is obtained by expression of a recombinant viral coat protein in host, preferably in a bacterial cell.

Immunostimulatory Nucleic Acid:

As used herein, the term immunostimulatory nucleic acid refers to a nucleic acid capable of inducing and/or enhancing an immune response. Immunostimulatory nucleic acids comprise ribonucleic acids and in particular desoxyribonucleic acids, wherein both, ribonucleic acids and desoxyribonucleic acids may be either double stranded or single stranded. Preferred IS S-NA are desoxyribonucleic acids, wherein further preferably said desoxyribonucleic acids are single stranded. Preferably, immunostimulatory nucleic acids contain at least one CpG motif comprising an unmethylated C. Very preferred immunostimulatory nucleic acids comprise at least one CpG motif, wherein said at least one CpG motif comprises or preferably consist of at least one, preferably one, CG dinucleotide, wherein the C is unmethylated. Preferably, but not necessarily, said CG dinucleotide is part of a palindromic sequence. The term immunostimulatory nucleic acid also refers to nucleic acids that contain modified bases, preferably 4-bromo-cytosine. Specifically preferred in the context of the invention are ISS-NA which are capable of stimulating IFN-alpha production in dendritic cells. Immunostimulatory nucleic acids useful for the purpose of the invention are described, for example, in WO2007/068747A1.

Oligonucleotide:

As used herein, the term “oligonucleotide” refers to a nucleic acid sequence comprising 2 or more nucleotides, preferably about 6 to about 200 nucleotides, and more preferably 20 to about 100 nucleotides, and most preferably 20 to 40 nucleotides. Very preferably, oligonucleotides comprise about 30 nucleotides, more preferably oligonucleotides comprise exactly 30 nucleotides, and most preferably oligonucleotides consist of exactly 30 nucleotides. Oligonucleotides are polyribonucleotides or polydeoxyribonucleotides and are preferably selected from (a) unmodified RNA or DNA, and (b) modified RNA or DNA. The modification may comprise the backbone or nucleotide analogues. Oligonucleotides are preferably selected from the group consisting of (a) single- and double-stranded DNA, (b) DNA that is a mixture of single- and double-stranded regions, (c) single- and double-stranded RNA, (d) RNA that is mixture of single- and double-stranded regions, and (e) hybrid molecules comprising DNA and RNA that are single-stranded or, more preferably, double-stranded or a mixture of single- and double-stranded regions. Preferred nucleotide modifications/analogs are selected from the group consisting of (a) peptide nucleic acid, (b) inosin, (c) tritylated bases, (d) phosphorothioates, (e) alkylphosphorothioates, (f) 5-nitroindole desoxyribofuranosyl, (g) 5-methyldesoxycytosine, and (h) 5,6-dihydro-5,6-dihydroxydesoxythymidine. Phosphothioated nucleotides are protected against degradation in a cell or an organism and are therefore preferred nucleotide modifications. Unmodified oligonucleotides consisting exclusively of phosphodiester bound nucleotides, typically are more active than modified nucleotides and are therefore generally preferred in the context of the invention. Most preferred are oligonucleotides consisting exclusively of phosphodiester bound deoxinucleotides, wherein further preferably said oligonucleotides are single stranded. Further preferred are oligonucleotides capable of stimulating IFN-alpha production in cells, preferably in dendritic cells. Very preferred oligonucleotides capable of stimulating IFN-alpha production in cells are selected from A-type CpGs and C-type CpGs.

CpG Motif:

As used herein, the term “CpG motif” refers to a pattern of nucleotides that includes an unmethylated central CpG, i.e. the unmethylated CpG dinucleotide, in which the C is unmethylated, surrounded by at least one base, preferably one or two nucleotides, flanking (on the 3′ and the 5′ side of) the central CpG. Typically and preferably, the CpG motif as used herein, comprises or alternatively consists of the unmethylated CpG dinucleotide and two nucleotides on its 5′ and 3′ ends. Without being bound by theory, the bases flanking the CpG confer a significant part of the activity to the CpG oligonucleotide.

Unmethylated CpG-Containing Oligonucleotide:

As used herein, the term “unmethylated CpG-containing oligonucleotide” or “CpG” refers to an oligonucleotide, preferably to an oligodesoxynucleotide, containing at least one CpG motif. Thus, a CpG contains at least one unmethylated cytosine, guanine dinucleotide. Preferred CpGs stimulate/activate, e.g. have a mitogenic effect on, or induce or increase cytokine expression by, a vertebrate bone marrow derived cell. For example, CpGs can be useful in activating B cells, NK cells and antigen-presenting cells, such as dendritic cells, monocytes and macrophages. Preferably, CpG relates to an oligodesoxynucleotide, preferably to a single stranded oligodesoxynucleotide, containing an unmethylated cytosine followed 3′ by a guanosine, wherein said unmethylated cytosine and said guanosine are linked by a phosphate bond, wherein preferably said phosphate bound is a phosphodiester bound or a phosphothioate bound, and wherein further preferably said phosphate bond is a phosphodiester bound. CpGs can include nucleotide analogs such as analogs containing phosphorothioester bonds and can be double-stranded or single-stranded. Generally, double-stranded molecules are more stable in vivo, while single-stranded molecules have increased immune activity. Preferably, as used herein, a CpG is an oligonucleotide that is at least about ten nucleotides in length and comprises at least one CpG motif, wherein further preferably said CpG is 10 to 60, more preferably 15 to 50, still more preferably 20 to 40, still more preferably about 30, and most preferably exactly 30 nucleotides in length. A CpG may consist of methylated and/or unmethylated nucleotides, wherein said at least one CpG motif comprises at least one CG dinucleotide wherein the C is unmethylated. The CpG may also comprise methylated and unmethylated sequence stretches, wherein said at least one CpG motif comprises at least one CG dinucleotide wherein the C is unmethylated. Very preferably, CpG relates to a single stranded oligodesoxynucleotide containing an unmethylated cytosine followed 3′ by a guanosine, wherein said unmethylated cytosine and said guanosine are linked by a phosphodiester bound. The CpGs can include nucleotide analogs such as analogs containing phosphorothioester bonds and can be double-stranded or single-stranded. Generally, phosphodiester CpGs are A-type CpGs as indicated below, while phosphothioester stabilized CpGs are B-type CpGs. Preferred CpG oligonucleotides in the context of the invention are A-type CpGs.

A-Type CpG:

As used herein, the term “A-type CpG” or “D-type CpG” refers to an oligodesoxynucleotide (ODN) comprising at least one CpG motif. A-type CpGs preferentially stimulate activation of T cells and the maturation of dendritic cells and are capable of stimulating IFN-alpha production. In A-type CpGs, the nucleotides of the at least one CpG motif are linked by at least one phosphodiester bond. A-type CpGs comprise at least one phosphodiester bond CpG motif which may be flanked at its 5′ end and/or, preferably and, at its 3′ end by phosphorothioate bound nucleotides. Preferably, the CpG motif, and hereby preferably the CG dinucleotide and its immediate flanking regions comprising at least one, preferably two nucleotides, are composed of phosphodiester nucleotides. Preferred A-type CpGs exclusively consist of phosphodiester (PO) bond nucleotides. Typically and preferably, the poly G motif comprises or alternatively consists of at least one, preferably at least three, at least 4, 5, 6, 7, 8, 9, 10, 11, 12, 13, 14, or 15 Gs (guanosines), most preferably by at least 10 Gs. Preferably, the A-type CpG of the invention comprises or alternatively consists of a palindromic sequence.

Palindromic Sequence:

A palindromic sequences is a nucleotide sequence which, when existing in the form of a double stranded nucleic acid with regular base pairing (A/T; C/G), would consist of two single strands with identical sequence in 5′-3′ direction.

Packaged:

The term “packaged” as used herein refers to the state of an immunostimulatory nucleic acid in relation to the VLP. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of an immunostimulatory nucleic acid. Thus, the immunostimulatory nucleic acid can be enclosed by the VLP without the existence of an actual binding, in particular of a covalent binding. In preferred embodiments, the immunostimulatory nucleic acid is packaged inside the VLP, most preferably in a non-covalent manner. In case said immunostimulatory nucleic acid is a DNA, preferably an unmethylated CpG-containing oligonucleotide, the term packaged implies that said immunostimulatory nucleic acid, preferably said unmethylated CpG-containing oligonucleotide, is not accessible to nucleases hydrolysis, preferably not accessible to DNAse hydrolysis (e.g. DNaseI or Benzonase), wherein preferably said accessibility is assayed as described in Examples 11-17 of WO2003/024481A2.

One, a, or an: when the terms “one”, “a”, or “an” are used in this disclosure, they mean “at least one” or “one or more” unless otherwise indicated.

In one aspect, the invention relates to a composition comprising: (a) a virus-like particle (VLP) with at least one first attachment site, wherein preferably said virus-like particle is a virus-like particle of an RNA bacteriophage; and (b) at least one antigen with at least one second attachment site, wherein said at least one antigen is an ectodomain of an influenza virus hemagglutinin protein (HA ectodomain) or a fragment of said ectodomain of an influenza virus hemagglutinin protein, wherein said fragment of said ectodomain of an influenza virus hemagglutinin protein comprises at least 80 contiguous amino acids of said ectodomain of an influenza virus hemagglutinin protein; and wherein (a) and (b) are linked through said at least one first and said at least one second attachment site.

In a preferred embodiment said HA ectodomain is a protein, wherein said protein is composed of (a) the HA1 subunit comprising or preferably consisting of amino acid position 11 to amino acid position 328 of SEQ ID NO:75 and (b) the HA2 subunit consisting of position 1 to 176 of SEQ ID NO:76.

In a further preferred embodiment said HA ectodomain is a HA ectodomain of influenza A virus, wherein preferably said influenza A virus belongs to a naturally occurring influenza A virus strain. In a further preferred embodiment said naturally occurring influenza A virus strain is selected from the group consisting of: (a) A/California/04/2009 (H1N1) (Genbank Accession No: ACP41105.1) (SEQ ID NO. 74); (b) A/Brisbane/59/2007 (H1N1) (Genbank Accession No: ACA28844.1) (SEQ ID NO. 73); (c) A/Albany/1/1968 (H2N2) (Genbank Accession No: AB052247.1); (d) A/northern shoveler/California/HKWF1128/2007 (H2N7) (Genbank Accession No: ACF_{47420.1}); (e) A/Uruguay/716/2007 X-175 (H3N2) (Genbank Accession No: ACD47234.1) (SEQ ID NO. 40); (f) A/ruddy turnstone/New Jersey/Sg-00542/2008 (H4N6) (Genbank Accession No: ACN86642.1); (g) A/Viet Nam/1203/2004 (H5N1) (Genbank Accession No: ABP51977.1) (SEQ ID NO. 41); (h) A/Indonesia/5/2005 (H5N1) (Genbank Accession No: ABWO6108.1) (SEQ ID NO. 42); (i) A/Egypt/2321-NAMRU3/2007 (H5N1) (Genbank Accession No: ABP96850.1) (SEQ ID NO. 43); (j) A/northern shoveler/California/HKWF383/2007 (H6N1) (Genbank Accession No: ACE76614.1); (k) A/Canada/rv504/2004 (H7N3) (Genbank Accession No: ABI85000.1); (l) A/duck/Mongolia/119/2008 (H7N9) (Genbank Accession No: BAH22785.1); (m) A/mallard/Minnesota/Sg-00570/2008 (H8N4) (Genbank Accession No: ACN86714.1); (n) A/HK/2108/2003 (H9N2) (Genbank Accession No: ABB58945.1); (o) A/Korea/KBNP-0028/2000 (H9N2) (Genbank Accession No: ABQ57378.1); (p) A/chicken/Anhui/AH16/2008 (H9N2) (Genbank Accession No: ACJ35235.1); (q) A/ruddy turnstone/New Jersey/Sg-00490/2008 (H10N7) (Genbank Accession No: ACN86516.1); (r) A/ruddy turnstone/New Jersey/Sg-00561/2008 (H11N9) (Genbank Accession No: ACN86684.1); (s) A/ruddy turnstone/New Jersey/Sg-00484/2008 (H12N5) (Genbank Accession No: ACN86498.1); (t) A/herring gull/Norway/10_{—}2336/2006 (H13N6) (Genbank Accession No: CAQ77191.1); (u) A/mallard duck/Astrakhan/263/1982 (H14N5) (Genbank Accession No: ABI84453.1); (v) A/Australian shelduck/Western Australia/1756/1983 (H15N2) (Genbank Accession No: ABB90704.1); (w) A/herring gull/Norway/10_{—}1623/2006 (H16N3) (Genbank Accession No: CAQ77189.1); (x) A/California/07/2009 (H1N1) (Genebank Accession No: ACP44189.1); and (y) A/Perth/16/2009 (H3N2) (Genebank Accession No: ACS71642.1). In a very preferred embodiment said naturally occurring influenza A virus strain is A/California/07/2009 (H1N1) (Genebank Accession No: ACP44189.1) or A/Perth/16/2009 (H3N2) (Genebank Accession No: ACS71642.1).

In a preferred embodiment of the present invention, said HA ectodomain is selected from the group consisting of the ectodomain of influenza A virus hemagglutinin protein subtype H1, H2, H3, H4, H5, H6, H7, H8, H9, H10, H11, H12, H13, H14, H15 and H16. Preferably, said HA ectodomain is selected from the group consisting of the ectodomain of influenza A virus hemagglutinin protein subtype H1, H2, H3, H5, H7 and H9, wherein more preferably, said HA ectodomain is selected from the group consisting of the ectodomain of influenza A virus hemagglutinin protein subtype H1, H2, H3, H5 and H9, wherein still more preferably said HA ectodomain is selected from the group consisting of the ectodomain of influenza A virus hemagglutinin protein subtype H1, H3, and H5. Further preferably said HA ectodomain is selected from the group consisting of the ectodomain of influenza A virus hemagglutinin protein subtype H1, H2, and H3. In a further preferred embodiment said HA ectodomain is the ectodomain of influenza A virus hemagglutinin protein subtype H1. In a further preferred embodiment said HA ectodomain is the ectodomain of influenza A virus hemagglutinin protein subtype H3. In a further preferred embodiment said HA ectodomain is the ectodomain of influenza A virus hemagglutinin protein subtype H3. In a further preferred embodiment said HA ectodomain is the ectodomain of influenza A virus hemagglutinin protein subtype H5.

In a further preferred embodiment the amino acid sequence of said ectodomain of said influenza A virus hemagglutinin protein is selected from the group consisting of: (i) the amino acid sequence as set forth in SEQ ID NO:39; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:39, wherein further preferably said ectodomain of said influenza A virus hemagglutinin protein is a naturally occurring ectodomain of influenza A virus hemagglutinin protein.

In a further preferred embodiment the amino acid sequence of said ectodomain of said influenza A virus hemagglutinin protein is selected from the group consisting of: (i) the amino acid sequence as set forth in SEQ ID NO:40; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:40, wherein further preferably said ectodomain of said influenza A virus hemagglutinin protein is a naturally occurring ectodomain of influenza A virus hemagglutinin protein.

In a further preferred embodiment the amino acid sequence of said ectodomain of said influenza A virus hemagglutinin protein is selected from the group consisting of: (i) the amino acid sequence as set forth in SEQ ID NO:41; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:41, wherein further preferably said ectodomain of said influenza A virus hemagglutinin protein is a naturally occurring ectodomain of influenza A virus hemagglutinin protein.

In a further preferred embodiment the amino acid sequence of said ectodomain of said influenza A virus hemagglutinin protein is selected from the group consisting of: (i) the amino acid sequence as set forth in SEQ ID NO:42; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:42, wherein further preferably said ectodomain of said influenza A virus hemagglutinin protein is a naturally occurring ectodomain of influenza A virus hemagglutinin protein.

In a further preferred embodiment the amino acid sequence of said ectodomain of said influenza A virus hemagglutinin protein is selected from the group consisting of: (i) the amino acid sequence as set forth in SEQ ID NO:43; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:43, wherein further preferably said ectodomain of said influenza A virus hemagglutinin protein is a naturally occurring ectodomain of influenza A virus hemagglutinin protein.

In a further preferred embodiment the amino acid sequence of said ectodomain of said influenza A virus hemagglutinin protein is selected from the group consisting of: (i) the amino acid sequence as set forth in SEQ ID NO:73; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:73, wherein further preferably said ectodomain of said influenza A virus hemagglutinin protein is a naturally occurring ectodomain of influenza A virus hemagglutinin protein.

In a further preferred embodiment the amino acid sequence of said ectodomain of said influenza A virus hemagglutinin protein is selected from the group consisting of: (i) the amino acid sequence as set forth in SEQ ID NO:74; and (ii) an amino acid sequence of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% amino acid sequence identity with SEQ ID NO:74, wherein further preferably said ectodomain of said influenza A virus hemagglutinin protein is a naturally occurring ectodomain of influenza A virus hemagglutinin protein.

In a further preferred embodiment said HA ectodomain is a HA ectodomain of influenza B virus, wherein preferably said influenza B virus belongs to a naturally occurring influenza B virus strain. In a preferred embodiment, said naturally occurring influenza B virus strain is selected from the group consisting of (a) B/Brisbane/33/2008 (Genbank Accession No: ACN29387.1); (b) B/Guangzhou/01/2007 (Genbank Accession No: ABX71684.1); and (c) B/Brisbane/60/2008 (Genbank Accession No: ACN29383.1).

In a further preferred embodiment said antigen is an ectodomain of an influenza virus hemagglutinin protein, wherein preferably said ectodomain of an influenza virus hemagglutinin protein is in a trimeric form. In a further preferred embodiment said trimeric form of said ectodomain of an influenza virus hemagglutinin protein is obtainable by a process comprising the steps of (i) recombinantly forming a construct by fusing a trimerization domain of bacteriophage T4 protein fibritin, or a functional fragment thereof, to said ectodomain of an influenza virus hemagglutinin protein, preferably the C-terminus of said ectodomain of an influenza virus hemagglutinin protein, (ii) expressing said construct in a eukaryotic or prokaryotic cell-based system, preferably in a baculovirus/insect cell system (iii) purifying said trimeric form. In a preferred embodiment said trimerization domain of bacteriophage T4 protein fibritin is SEQ ID NO:95, or a functional fragment thereof. In a very preferred embodiment said trimerization domain of bacteriophage T4 protein fibritin is SEQ ID NO:95. The expression of the constructs is preferably performed in Hi5 or sf21 insect cells preferably sf21 insect cells. The antigen may further incorporate a His-tag at the C-terminus of the said ectodomain of the influenza virus hemagglutinin protein to enable purification. The said His-tag preferably comprises 3 to 6 histidine residues, preferably 6 histidine residues fused to the C-terminus of said ectodomain of the influenza virus hemagglutinin protein containing the trimerizing sequence, preferably to the C-terminus of said ectodomain of the influenza virus hemagglutinin.

In a further preferred embodiment said antigen is a fragment of said HA ectodomain, wherein preferably said fragment of said HA ectodomain is the HA1 subunit of said HA ectodomain or a fragment of said HA1 subunit of said HA ectodomain.

In a further preferred embodiment said fragment of said HA ectodomain comprises or preferably consists of an amino acid sequence corresponding to position 11 to position 328 of SEQ ID NO:75. In a further preferred embodiment said fragment of said HA ectodomain consists of an amino acid sequence corresponding to position 11 to position 329 of SEQ ID NO:75. In a further preferred embodiment said fragment of said HA ectodomain comprises, or preferably consists of, an amino acid sequence corresponding to position 115 to position 261 of SEQ ID NO:75. In a further preferred embodiment said fragment of said HA ectodomain comprises, or preferably consists of, an amino acid sequence corresponding to position 50 to position 261 of SEQ ID NO:75. In a further preferred embodiment said fragment of said HA ectodomain comprises the amino acid residues tyrosine corresponding to the positions 98 and 195 of SEQ ID NO:75, tryptophan corresponding to the position 153 of SEQ ID NO:75, and histidine corresponding to the position 183 of SEQ ID NO:75.

In a further preferred embodiment, said fragment of said HA ectodomain comprises at least one disulphide bond, preferably at least 2 disulphide bonds, more preferably at least 3, and still more preferably at least 4 disulphide bonds. Thus, in a further preferred embodiment said fragment of said HA ectodomain comprises a cysteine residue corresponding to positions 97 and 139 of SEQ ID NO:75, preferably said fragment of said HA ectodomain comprises a cysteine residue corresponding to positions 64, 76, 97, 139 of SEQ ID NO:75, more preferably said fragment of said HA ectodomain comprises a cysteine residue corresponding to positions 52, 64, 76, 97, 139, 277, 281, 305 of SEQ ID NO:75.

In a further preferred embodiment said fragment of said HA ectodomain is a fragment of the HA1 subunit of said HA ectodomain. In a further preferred embodiment said fragment of said HA ectodomain comprises, or preferably consists of, an amino acid sequence corresponding to position 57 to position 270 of SEQ ID NO:75. In a further preferred embodiment said fragment of said HA ectodomain comprises, or preferably consists of, an amino acid sequence corresponding to position 57 to position 276 of SEQ ID NO:75.

In a further preferred embodiment said fragment of said HA ectodomain comprises, or preferably consists of, an amino acid sequence corresponding to position 46 to position 310 of SEQ ID NO:75. In a further preferred embodiment said fragment of said HA ectodomain comprises, or preferably consists of, an amino acid sequence corresponding to position 46 to position 310 of SEQ ID NO:75, wherein said HA ectodomain has an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with the HA ectodomain of influenza A virus strain A/California/07/2009 (H1N1) (Genebank Accession No: ACP44189.1) or A/Perth/16/2009 (H3N2) (Genebank Accession No: ACS71642.1), and wherein preferably said HA ectodomain is a naturally occurring HA ectodomain.

In a further preferred embodiment said fragment of said HA ectodomain comprises, or preferably consists of, an amino acid sequence corresponding to position 46 to position 310 of SEQ ID NO:75, wherein said HA ectodomain has an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with the HA ectodomain of influenza B virus strain B/Brisbane/33/2008 (Genbank Accession No: ACN29387.1), B/Guangzhou/01/2007 (Genbank Accession No: ABX71684.1), or B/Brisbane/60/2008 (Genbank Accession No: ACN29383.1), and wherein preferably said HA ectodomain is a naturally occurring HA ectodomain.

In a further preferred embodiment said fragment of said HA ectodomain comprises, or preferably consists of, an amino acid sequence corresponding to position 42 to position 310 of SEQ ID NO:75. In a further preferred embodiment said fragment of said HA ectodomain comprises, or preferably consists of, an amino acid sequence corresponding to position 42 to position 310 of SEQ ID NO:75, wherein said HA ectodomain has an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with the HA ectodomain of influenza A virus strain A/California/07/2009 (H1N1) (Genebank Accession No: ACP44189.1) or A/Perth/16/2009 (H3N2) (Genebank Accession No: ACS71642.1), and wherein preferably said HA ectodomain is a naturally occurring HA ectodomain.

In a further preferred embodiment said fragment of said HA ectodomain comprises, or preferably consists of, an amino acid sequence corresponding to position 42 to position 310 of SEQ ID NO:75, wherein said HA ectodomain has an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with the HA ectodomain of influenza B virus strain B/Brisbane/33/2008 (Genbank Accession No: ACN29387.1), B/Guangzhou/01/2007 (Genbank Accession No: ABX71684.1), or B/Brisbane/60/2008 (Genbank Accession No: ACN29383.1), and wherein preferably said HA ectodomain is a naturally occurring HA ectodomain.

In a further preferred embodiment said fragment of said HA ectodomain comprises, or preferably consists of, an amino acid sequence corresponding to position 54 to position 276 of SEQ ID NO:75. In a further preferred embodiment said fragment of said HA ectodomain comprises, or preferably consists of, an amino acid sequence corresponding to position 54 to position 270 of SEQ ID NO:75. In a further preferred embodiment said fragment of said HA ectodomain comprises, or preferably consists of, an amino acid sequence corresponding to 54a to position 276 of SEQ ID NO:75. In a further preferred embodiment said fragment of said HA ectodomain comprises, or preferably consists of, an amino acid sequence corresponding to 54a to position 270 of SEQ ID NO:75.

In a further preferred embodiment the amino acid sequence of said fragment of said HA ectodomain is an amino acid sequence having at least 90%, preferably at least 95%, more preferably at least 98%, and most preferably at least 99% amino acid sequence identity with an amino acid sequence selected from the group consisting of: (a) position 2 to 277 of SEQ ID NO:67; (b) position 2 to 273 of SEQ ID NO:68; (c) position 2 to 230 of SEQ ID NO:69; (d) position 2 to 230 of SEQ ID NO:70; (e) position 2 to 224 of SEQ ID NO:71; (f) position 2 to 221 of SEQ ID NO:72; (g) SEQ ID NO:84; (h) SEQ ID NO:85; (i) SEQ ID NO:86; (j) SEQ ID NO:88; (k) SEQ ID NO:89; and (l) SEQ ID NO:90.

In a further preferred embodiment the amino acid sequence of said fragment of said HA ectodomain is an amino acid sequence selected from the group consisting of: (a) position 2 to 277 of SEQ ID NO:67; (b) position 2 to 273 of SEQ ID NO:68; (c) position 2 to 230 of SEQ ID NO:69; (d) position 2 to 230 of SEQ ID NO:70; (e) position 2 to 224 of SEQ ID NO:71; and (f) position 2 to 221 of SEQ ID NO:72; (g) SEQ ID NO:84; (h) SEQ ID NO:85; (i) SEQ ID NO:86; (j) SEQ ID NO:88; (k) SEQ ID NO:89; and (l) SEQ ID NO:90.

In a further preferred embodiment the amino acid sequence of said fragment of said HA ectodomain is an amino acid sequence having at least 90%, preferably at least 95%, more preferably at least 98%, and most preferably at least 99% amino acid sequence identity with SEQ ID NO:87. In a further preferred embodiment the amino acid sequence of said fragment of said HA ectodomain is SEQ ID NO:87.

In a further preferred embodiment said at least one antigen with at least one second attachment site further comprises a linker, wherein said linker comprises or consists of said second attachment site. In a preferred embodiment said linker is associated to said antigen by way of one peptide bond, wherein preferably said linker is selected from the group consisting of (a) a cysteine residue; (b) CGG, and (c) GGC. Said at least one antigen with at least one second attachment site may further incorporate a His-tag at the C-terminus of the said ectodomain of the influenza virus hemagglutinin protein.

Thus, in a further preferred embodiment said at least one antigen with at least one second attachment site comprises or preferably consists of any one of SEQ ID NOs 67 to 72. It is hereby understood by the artisan, that the N-terminal methionine residue of the recombinantly produced polypeptide may be cleaved of. Thus, in a further preferred embodiment said at least one antigen comprises any one of SEQ ID NOs 84 to 90.

In a preferred embodiment the composition of the invention is capable of inducing hemagglutination of red blood cells at a concentration of less than 0.50 μg of said composition in 1 μl of 1% red blood cells. The hemagglutination assay is hereby preferably performed under conditions as described in Example 35.

The present invention preferably relates to virus-like particles of viruses which are disclosed on p. 46-52 of WO2007/068747A1, which is incorporated herewith by way of reference. In a preferred embodiment, the VLP is a recombinant VLP. A recombinant VLP is obtained by expressing the coat protein in a host cell, preferably in a bacterial cell, most preferably in E. coli.

In a further preferred embodiment the VLP is a VLP of an RNA bacteriophage. The present invention preferably relates to virus-like particles of RNA bacteriophages disclosed on pages 49-50 of WO2007/068747A1, which is incorporated herewith by way of reference.

It is a specific advantage of coat proteins of RNA bacteriophages that they can readily be expressed in bacterial expression systems, in particular in E. coli. Thus, in one preferred embodiment of the invention, the virus-like particle comprises, consists essentially of, or alternatively consists of, recombinant coat proteins of an RNA bacteriophage. Preferred coat proteins of RNA bacteriophages are the coat proteins disclosed as SEQ ID NOs 3 to 23 of WO2007/068747A1. In a preferred embodiment, the virus-like particle comprises, consists essentially of, or alternatively consists of, recombinant coat proteins, wherein preferably said recombinant coat proteins are recombinant coat proteins of an RNA bacteriophage. In a further preferred embodiment the virus-like particle comprises, consists essentially of, or alternatively consists of, recombinant coat proteins of RNA bacteriophage Qβ, of RNA bacteriophage AP205, or of RNA bacteriophage φCb5. In a further preferred embodiment the virus-like particle comprises, consists essentially of, or alternatively consists of, recombinant coat proteins comprising or preferably consisting of an amino acid sequence selected from the group consisting of: (a) SEQ ID NO:1 (Qβ coat protein); (b) a mixture of SEQ ID NO:1 and SEQ ID NO:2 (Qβ A1 protein); (c) SEQ ID NO:19 (AP205 coat protein); (d) SEQ ID NO:92 (φCb5 R21); (e) SEQ ID NO:93 (φCb5 K21); and (f) SEQ ID NO:94 (φCb5 K21 double Cys).

In one preferred embodiment, the VLP is a VLP of RNA bacteriophage Qβ. Thus, in a further preferred embodiment the virus-like particle comprises, consists essentially of, or alternatively consists of, recombinant coat proteins of RNA bacteriophage Qβ. In a further preferred embodiment the virus-like particle comprises, consists essentially of, or alternatively consists of, recombinant coat proteins comprising or preferably consisting of SEQ ID NO:1. Further preferred virus-like particles of RNA bacteriophages, in particular of bacteriophage Qβ and bacteriophage fr, are disclosed in WO 02/056905, the disclosure of which is herewith incorporated by reference in its entirety. In particular Example 18 of WO 02/056905 contains a detailed description of the preparation of VLP particles of bacteriophage Qβ.

In a further preferred embodiment, the VLP is a VLP of bacteriophage AP205. Thus, in a further preferred embodiment the virus-like particle comprises, consists essentially of, or alternatively consists of, recombinant coat proteins of RNA bacteriophage AP205. In a further preferred embodiment the virus-like particle comprises, consists essentially of, or alternatively consists of, recombinant coat proteins comprising or preferably consisting of SEQ ID NO:19. Further preferred VLPs of bacteriophage AP205 are those described in WO2004/007538, in particular in Example 1 and Example 2 therein.

In a further preferred embodiment, the VLP is a VLP of RNA bacteriophage φCb5. Thus, in a further preferred embodiment the virus-like particle comprises, consists essentially of, or alternatively consists of, recombinant coat proteins of RNA bacteriophage φCb5. In a further preferred embodiment the virus-like particle comprises, consists essentially of, or alternatively consists of, recombinant coat proteins comprising or preferably consisting of any one of SEQ ID NOs 92 to 94, preferably SEQ ID NO:92.

In a further aspect, the invention relates to a method of producing the compositions of the invention comprising (a) providing a virus-like particle with at least one first attachment site, wherein said virus-like particle is a virus-like particle of an RNA bacteriophage; (b) providing at least one antigen with at least one second attachment site, wherein said at least one antigen is an ectodomain of an influenza virus hemagglutinin protein or a fragment of said ectodomain of an influenza virus hemagglutinin protein, wherein said fragment of said ectodomain of an influenza virus hemagglutinin protein comprises at least 80 contiguous amino acids of said ectodomain of an influenza virus hemagglutinin protein; and (c) combining said virus-like particle and said at least one antigen to produce said composition, wherein said at least one antigen and said virus-like particle are linked through the first and the second attachment sites. In a preferred embodiment, the provision of the at least one antigen with the at least one second attachment site is by way of expression, preferably by way of expression in a bacterial system, preferably in E. coli.

In one preferred embodiment, the said virus-like particle with at least one first attachment site and said at least one antigen with said at least one second attachment site are linked via at least one peptide covalent bond. A gene encoding said antigen is in-frame ligated, either internally or preferably to the N- or the C-terminus to the gene encoding a coat protein, wherein the fusion protein preferably retains the ability of forming a virus-like particle. Further embodiments encompass fusion of the antigen to coat protein sequences as described in Kozlovska, T. M., et al., Intervirology 39:9-15 (1996), Pushko P. et al., Prot. Eng. 6:883-891 (1993), WO 92/13081), or in U.S. Pat. No. 5,698,424.

In a further preferred embodiment said virus-like particle with at least one first attachment site and said at least one antigen with said at least one second attachment site are linked via at least one non-peptide covalent bond. In a further preferred embodiment said first attachment site and said second attachment site are linked via at least one non-peptide covalent bond.

Attachment between capsids and antigenic proteins by way of disulfide bonds are labile, in particular, to sulfhydryl-moiety containing molecules, and are, furthermore, less stable in serum than, for example, thioether attachments (Martin F J. and Papahadjopoulos D. (1982), Irreversible Coupling of Immunoglobulin Fragments to Preformed Vesicles. J. Biol. Chem. 257: 286-288). Therefore, in a further very preferred embodiment, the association or linkage between said virus-like particle with at least one first attachment site and said at least one antigen with said at least one second attachment site does not comprise a a sulphur-sulphur bond. In a further very preferred embodiment, said at least one first attachment site is not or does not comprise a sulfhydryl group. In again a further very preferred embodiment, said at least one first attachment site is not or does not comprise a sulfhydryl group of a cysteine.

In a preferred embodiment, the first attachment site comprises, or preferably is, an amino group, preferably the amino group of a lysine residue, wherein preferably said lysine residue is a lysine residue comprised by a coat protein of said virus-like particle, and wherein further preferably said lysine residue is a lysine residue comprised by a recombinant coat protein of an RNA bacteriophage, most preferably of RNA bacteriophage Qβ, of RNA bacteriophage AP205, or of RNA bacteriophage φCb5. In a very preferred embodiment said lysine residue is a lysine residue of SEQ ID NO:1, 19, or of any one of SEQ ID NOs 92 to 93. In another preferred embodiment, the second attachment site comprises, or preferably is, a sulfhydryl group, preferably a sulfhydryl group of a cysteine.

In a further preferred embodiment said at least one first attachment comprises an amino group and said second attachment comprises a sulfhydryl group. In a further preferred embodiment, said first attachment is an amino group and said second attachment site is a sulfhydryl group. In a still further preferred embodiment, said first attachment is an amino group of a lysine residue, wherein preferably said lysine residue is a lysine residue comprised by a coat protein of said virus-like particle, and said second attachment site is a sulfhydryl group of a cysteine residue.

In a further preferred embodiment said virus-like particle with at least one first attachment site comprises, consists essentially of, or alternatively consists of a recombinant coat protein of an RNA bacteriophage, wherein said recombinant coat proteins comprise or preferably consist of the amino acid sequence of SEQ ID NO:1, 19, or any one of SEQ ID NOs 92 to 94, and wherein said first attachment site comprises, or preferably is, an amino group of a lysine residue of said amino acid sequence. In a further preferred embodiment said recombinant coat proteins comprise or preferably consist of the amino acid sequence of SEQ ID NO:1 and said first attachment site comprises, or preferably is, an amino group of a lysine residue of SEQ ID NO:1.

In a further preferred embodiment only one of said second attachment sites associates with said first attachment site through at least one non-peptide covalent bond leading to a single and uniform type of binding of said antigen to said virus-like particle, wherein said only one second attachment site that associates with said first attachment site is a sulfhydryl group, and wherein said antigen and said virus-like particle interact through said association to form an ordered and repetitive antigen array.

Linking of the antigen to the VLP by using a hetero-bifunctional cross-linker allows coupling of the antigen to the VLP in an oriented fashion. Thus, in one preferred embodiment said virus-like particle with at least one first attachment site and said at least one antigen with said at least one second attachment site are linked by way of chemical cross-linking, typically and preferably by using a hetero-bifunctional cross-linker. In preferred embodiments, the hetero-bifunctional cross-linker comprises (a) a functional group which reacts with the preferred first attachment site, preferably with an amino group, more preferably with an amino group of a lysine residue, of the VLP, and (b) a further functional group which reacts with the preferred second attachment site, preferably with a sulfhydryl group, most preferably with a sulfhydryl group of a cysteine residue, which is inherent of, or artificially added to the antigen, and optionally also made available for reaction by reduction. Thus, preferred hetero-bifunctional cross-linkers comprise one functional group reactive towards amino groups and one functional group reactive towards sulfhydryl groups. Very preferred hetero-bifunctional cross-linkers are selected from the group consisting of SMPH (Pierce), Sulfo-MBS, Sulfo-EMCS, Sulfo-GMBS, Sulfo-SIAB, Sulfo-SMPB, Sulfo-SMCC, Sulfo-KMUS, SVSB, and SIA, wherein most preferably said hetero-bifunctional cross-linker is SMPH. The above mentioned cross-linkers all lead to formation of an amide bond after reaction with the amino group and a thioether linkage with the sulfhydryl groups.

In a preferred embodiment said at least one antigen with at least one second attachment site further comprises a linker, wherein preferably said linker comprises or consists of said second attachment site. In a preferred embodiment, said linker associates said at least one first and said at least one second attachment site. In a further preferred embodiment of the present invention, a linker is associated to the antigen by way of at least one covalent bond, preferably, by at least one, preferably one peptide bond. In a further preferred embodiment said at least one antigen with said at least one second attachment site comprises a linker, wherein said linker comprises said second attachment site, and wherein preferably said linker is associated to said antigen by way of one peptide bond, and wherein further preferably said linker comprises or alternatively consists of a cysteine residue. Preferably, the linker comprises, or alternatively consists of, the second attachment site. In a further preferred embodiment, the linker comprises a sulfhydryl group, preferably a cysteine residue. In another preferred embodiment, the linker comprises or preferably is a cysteine residue. In a further preferred embodiments said linker is selected from the group consisting of: (a) CGG; (b) N-terminal glycine linkers, preferably GCGGGG; (c) GGC; and (d) C-terminal glycine linkers, preferably GGGGCG. Further linkers useful for the invention are disclosed, for example, in WO2007/039552A1 (p. 32, paragraphs 111 and 112). In a preferred embodiment, the linker is added to the C-terminus of the antigen.

In a further preferred embodiment said composition further comprises at least one immunostimulatory substance. Immunostimulatory substances useful for the invention are generally known in the art and are disclosed, inter alia, in WO2003/024481A2.

In a further preferred embodiment said immunostimulatory substance is bound to said virus-like particle. In a further preferred embodiment said immunostimulatory substance is mixed with said virus-like particle. In a further preferred embodiment said immunostimulatory substance is selected from the group consisting of: (a) immunostimulatory nucleic acid; (b) peptidoglycan; (c) lipopolysaccharide; (d) lipoteichonic acid; (e) imidazoquinoline compound; (f) flagelline; (g) lipoprotein; and (h) any mixtures of at least one substance of (a) to (g).

In a further preferred embodiment said immunostimulatory substance is an immunostimulatory nucleic acid, wherein preferably said immunostimulatory nucleic acid is selected from the group consisting of: (a) ribonucleic acids; (b) deoxyribonucleic acids; (c) chimeric nucleic acids; and (d) any mixture of (a), (b) and/or (c).

In a further preferred embodiment said immunostimulatory nucleic is a ribonucleic acid, and wherein said ribonucleic acid is host cell derived RNA. In a further preferred embodiment said immunostimulatory nucleic is poly-(I:C) or a derivative thereof.

In a further preferred embodiment said immunostimulatory nucleic is a deoxyribonucleic acid, wherein preferably said deoxyribonucleic acid is an unmethylated CpG-containing oligonucleotide. In a further preferred embodiment said unmethylated CpG-containing oligonucleotide is an A-type CpG.

In a further preferred embodiment, said immunostimulatory nucleic acid, and hereby preferably said deoxyribonucleic acid, and hereby still further preferably said unmethylated CpG-containing oligonucleotid, is packaged into said virus-like particle.

In a further preferred embodiment said unmethylated CpG-containing oligonucleotide comprises a palindromic sequence. In a further preferred embodiment the CpG motif of said unmethylated CpG-containing oligonucleotide is part of a palindromic sequence. In a further preferred embodiment said palindromic sequence is GACGATCGTC (SEQ ID NO:96).

In a further preferred embodiment said palindromic sequence is flanked at its 5′-terminus and at its 3′-terminus by guanosine entities. In a further preferred embodiment said palindromic sequence is flanked at its 5′-terminus by at least 3 and at most 15 guanosine entities, and wherein said palindromic sequence is flanked at its 3′-terminus by at least 3 and at most 15 guanosine entities. In a further preferred embodiment said unmethylated CpG-containing oligonucleotide comprises or alternatively consists of the sequence selected from the group consisting of: (a) “G6-6” GGGGGGGACGATCGTCGGGGGG (SEQ ID NO:97); (b) “G7-7” GGGGGGGGACGATCGTCGGGGGGG (SEQ ID NO:98); (c) “G8-8” GGGGGGGGGACGATCGTCGGGGGGGG (SEQ ID NO:99); (d) “G9-9” GGGGGGGGGGACGATCGTCGGGGGGGGG (SEQ ID NO:100); and (e) “G10” GGGGGGGGGGGACGATCGTCGGGGGGGGGG (SEQ ID NO:101). In a further preferred embodiment said unmethylated CpG-containing oligonucleotide comprises or alternatively consists of the sequence GGGGGGGGGGGACGATCGTCGGGGGGGGGG (SEQ ID NO:101). In a further preferred embodiment said unmethylated CpG-containing oligonucleotide consists exclusively of phosphodiester bound nucleotides, wherein preferably said unmethylated CpG-containing oligonucleotide is packaged into said VLP.

In a further preferred embodiment said immunostimulatory nucleic acid, preferably said unmethylated CpG-containing oligonucleotide, is not accessible to DNAse hydrolysis. In a further preferred embodiment said immunostimulatory nucleic acid is an unmethylated CpG-containing oligonucleotide, wherein said unmethylated CpG-containing oligonucleotide is not accessibly to Benzonase hydrolysis. In a further preferred embodiment said immunostimulatory nucleic acid is an unmethylated CpG containing oligonucleotide consisting of the sequence GGGGGGGGGGGACGATCGTCGGGGGGGGGG (SEQ ID NO:101), wherein said unmethylated CpG-containing oligonucleotide consists exclusively of phosphodiester bound nucleotides, and wherein preferably said unmethylated CpG containing oligonucleotide is packaged into said VLP.

A further aspect of the invention is a vaccine composition comprising or preferably consisting of a composition of the invention, wherein preferably said vaccine composition comprises an effective amount of the composition of the invention, and wherein further preferably said vaccine composition comprises a therapeutically effective amount of the composition of the invention. An “effective amount” hereby refers to an amount that produces the desired physiological, preferably immunological effect. A “therapeutically effective amount” hereby refers to an amount that produces the desired therapeutic effect. In the context of the invention the desired therapeutic effect is the prevention or the amelioration of an influenza virus infection in an animal, preferably in a human.

An advantageous feature of the present invention is the high immunogenicity of the composition, even in the absence of adjuvants. Therefore, in a preferred embodiment, the vaccine composition is devoid of adjuvant. The absence of an adjuvant, furthermore, minimizes the occurrence of unwanted side effects. Thus, the administration of the vaccine composition to a patient will preferably occur without administering adjuvant to the same patient prior to, simultaneously or after the administration of the vaccine composition.

In a further preferred embodiment, the vaccine composition further comprises at least one adjuvant. When an adjuvant is administered, the administration of the at least one adjuvant may hereby occur prior to, simultaneously or after the administration of the inventive composition or of the vaccine composition.

A further aspect of the invention is a pharmaceutical composition comprising: (1) a composition or a vaccine composition of the invention; and (2) a pharmaceutically acceptable carrier or excipient. The composition and/or the vaccine composition of the invention is administered to an individual in a pharmaceutically acceptable form. The pharmaceutical composition of the invention is said to be pharmaceutically acceptable if their administration can be tolerated by a recipient individual, preferably by a human. A pharmaceutically acceptable carrier or excipient may contains salts, buffers, adjuvants, or other substances which are desirable for improving the efficacy of the conjugate. Examples of materials suitable for use in preparation of vaccine compositions or pharmaceutical compositions are provided, for example, in Remington's Pharmaceutical Sciences (Osol, A, ed., Mack Publishing Co., (1990)). This includes sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

In a further aspect the invention relates to a method of immunization, preferably to a method of immunization against influenza, most preferably against flu, said method comprising administering the composition, the vaccine composition, or the pharmaceutical composition of the invention to an animal, preferably to a human.

In a further aspect the invention relates to a method of treating, ameliorating and/or preventing influenza virus infection, preferably influenza A virus infection, in an animal, preferably in a human, said method comprising administering the composition, the vaccine composition, or the pharmaceutical composition of the invention to said animal, preferably to said human.

In a further aspect the invention relates to the composition, the vaccine composition, or the pharmaceutical composition of the invention for use as a medicament.

In a further aspect the invention relates to the composition, the vaccine composition, or the pharmaceutical composition of the invention for use in a method of treating, ameliorating and/or preventing influenza virus infection, preferably of influenza A virus infection.

In a further aspect the invention relates to a method of treatment, amelioration and/or prevention of influenza, preferably of influenza A, said method comprising administering a composition, a vaccine composition or a pharmaceutical composition of the invention to an animal, preferably to a human, wherein preferably said composition, said vaccine composition and/or said pharmaceutical composition are administered to said animal, more preferably to said human, in an effective amount, preferably in an immunologically effective amount. An immunologically effective amount hereby refers to an amount which is capable of raising a detectable immune response, preferably antibody response in said individual, preferably in said human.

In one embodiment, the compositions, vaccine compositions and/or pharmaceutical compositions are administered to said animal, preferably to said human by injection, infusion, inhalation, oral administration, or other suitable physical methods. In a preferred embodiment, the compositions, vaccine compositions and/or pharmaceutical compositions are administered to said animal, preferably to said human, intramuscularly, intravenously, transmucosally, transdermally, intranasally, intraperitoneally, subcutaneously, or directly into the lymph node.

In a further aspect the invention relates to the use of the compositions, of the vaccine compositions and/or of the pharmaceutical compositions of the invention for the treatment, amelioration and/or prevention of influenza, preferably of influenza A.

A further aspect of the invention is the use of the compositions, of the vaccine compositions and/or of the pharmaceutical compositions of the invention for the manufacture of a medicament for the treatment, amelioration and/or prevention of influenza, preferably of influenza A.

In a further aspect the invention relates to an antigen, wherein said antigen is a HA ectodomain or a fragment of a HA ectodomain as defined herein. In a preferred embodiment said antigen is a fragment of a HA ectodomain as defined herein. In a further preferred embodiment said antigen is a fragment of a HA ectodomain comprising, or preferably consisting of, an amino acid sequence corresponding to position 42 to position 310 of SEQ ID NO:75. In a further preferred embodiment said antigen is a fragment of a HA ectodomain comprising, or preferably consisting of, an amino acid sequence corresponding to position 42 to position 310 of SEQ ID NO:75, wherein said HA ectodomain has an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with the HA ectodomain of influenza A virus strain A/California/07/2009 (H1N1) (Genebank Accession No: ACP44189.1) or A/Perth/16/2009 (H3N2) (Genebank Accession No: ACS71642.1), and wherein preferably said HA ectodomain is a naturally occurring HA ectodomain.

In a further preferred embodiment said antigen is a fragment of a HA ectodomain comprising, or preferably consisting of, an amino acid sequence corresponding to position 42 to position 310 of SEQ ID NO:75, wherein said HA ectodomain has an amino acid sequence identity of at least 70%, preferably of at least 80%, more preferably of at least 80%, still more preferably of at least 85%, still more preferably of at least 90%, still more preferably of at least 95%, still more preferably of at least 96%, still more preferably of at least 97%, still more preferably of at least 98%, and most preferably of at least 99% with the HA ectodomain of influenza B virus strain B/Brisbane/33/2008 (Genbank Accession No: ACN29387.1), B/Guangzhou/01/2007 (Genbank Accession No: ABX71684.1), or B/Brisbane/60/2008 (Genbank Accession No: ACN29383.1), and wherein preferably said HA ectodomain is a naturally occurring HA ectodomain.

It is to be understood that all technical features and embodiments described herein, in particular those described for the compositions of the invention and its components, may be applied to all aspects of the invention, especially to the vaccine compositions, to the pharmaceutical compositions, to the methods and uses, alone or in any possible combination.

### EXAMPLES

### Example 1

### Cloning, Expression and Purification of ecHA A/PR/8/34 (H1N1)

A) Generation of pFastBac1_GP67

The vector pFastBac1_GP67 (SEQ ID NO:33) is a derivative of pFastBac1 (Invitrogen), in which the signal peptide of GP67 was introduced in front of the multiple cloning site for secretion of proteins. The vector was constructed by ligating the annealed pair of oligos PH155 (SEQ ID NO:20) and PH156 (SEQ ID NO:21) and the annealed pair of oligos PH157 (SEQ ID NO:22) and PH158 (SEQ ID NO:23) and the annealed pair of oligos PH159 (SEQ ID NO:24) and PH160 (SEQ ID NO:25) and the annealed pair of oligos PH161 (SEQ ID NO:26) and PH162 (SEQ ID NO:27) together into the BamHI-EcoRI digested pFastBac1 plasmid to obtain pFastBac1_GP67. The resulting plasmid has BamHI, EcoRI, PstI, XhoI, SphI, Acc65I, KpnI and HindIII restriction sites in its multiple cloning site.

B) Cloning and Sequencing of ecHA of Mouse Adapted Influenza A/PR8/34 (H1N1)

The cDNA of HA0 of (HA0 PR8) strain was produced by reverse transcription of vRNAs (−) extracted from the supernatant of influenza A PR8 infected MDCK cells using the primer Uni12 (SEQ ID NO:28) followed by PCR using the primers BM-HA-1 (SEQ ID NO:29) and BM-NS-890R (SEQ ID NO:30). The translated sequence of the ecHA from PR8 is SEQ ID NO:39.

C) Generation of pFastBac1_GP67_HA_PR8

A DNA encoding amino acids 11-329 (HA1) followed by amino acid 1-176 (HA2) [HA amino acid positions are based on H3 numbering] from mouse adapted PR8 (see under B) followed by a trimerizing sequence (foldon) from the bacteriophage T4 fibritin, a 6×His-tag and a cysteine containing linker was optimized for expression in mammalian cells and produced by gene synthesis (Geneart, Regensburg, Germany). The optimized nucleotide sequence was amplified with oligonucleotides PH163 (SEQ ID NO:31) and PH164 (SEQ ID NO:32). The resulting DNA fragment was digested with BamHI and XhoI and cloned into the BamHI-XhoI digested expression vector pFastBac1_GP67 resulting in plasmid pFastBac1_GP67_HA_PR8 (SEQ ID NO:34). This plasmid encodes for a fusion protein consisting of an N-terminus containing HA0 from mouse adapted PR8 (composed of aa 11-329 from HA1 fused to the N-terminus of aa 1-176 from HA2, aa positions of HA1 and HA2 are based on H3 numbering) (SEQ ID NO:39) fused to the N-terminus of SEQ ID NO:44. The fusion protein of SEQ ID NO:34 fused to the N-terminus of SEQ ID NO:44 was termed ecHA-PR8.

D) Generation of Recombinant Baculovirus, Production and Purification of ecHA

A recombinant baculovirus expressing ecHA-PR8 was generated using the Bac-to-Bac Baculovirus Expression System (Invitrogen) with plasmid pFastBac1_GP67_HA_PR8. For expression, Hi5 insect cells (Invitrogen) were grown at 27° C. and infected with recombinant baculovirus at an MOI of 5 and incubated for 72 h. The supernatant containing the recombinantly expressed protein ecHA-PR8 was harvested 72 h post infection (p.i.). The supernatant was concentrated 10 times by TFF using a GE hollow fiber cartridge UFP-5-C-35; 5,000 NMWC. Concentrated supernatant was applied to a Ni²⁺-NTA agarose column (Qiagen, Hilden, Germany). After extensive washing of the column with washing buffer (50 mM NaH₂PO₄, 300 mM NaCl, 20 mM Imidazol, pH 8.0) the protein was eluted with elution buffer (50 mM NaH₂PO₄, 300 mM NaCl, 200 mM Imidazol, pH 8.0). The purified protein was dialysed against PBS pH 7.2 and stored at −80° C. until further use.

### Example 2

### Cloning, Expression and Purification of ecHA from A/Uruguay/716/2007 X-175 (H3N2)

A DNA encoding amino acids 11-329 (HA1) followed by amino acid 1-176 (HA2) [HA amino acid positions are based on H3 numbering] from A/Uruguay/716/2007 X-175 (H3N2) (NCBI accession number ACD47234.1) flanked at the 3′ end by a BamHI restriction site and at the 5′ end by a AscI restriction site was optimized for expression in insect cells and produced by gene synthesis (Geneart, Regensburg, Germany). The resulting DNA fragment was digested with BamHI and AscI (SEQ ID NO:35) and cloned into the BamHI-AscI digested expression vector pFastBac1_GP67 HA_PR8 (described in EXAMPLE 1) resulting in plasmid pFastBac1_GP67_HA_A/Uruguay/716/2007 NYMC X-175C shortly termed pFastBac1_GP67_HA_A_Uruguay. This plasmid encodes for fusion protein consisting of an N-terminus containing HA0 from influenza A/Uruguay/716/2007 X-175 (composed of aa 11-329 from HA1 fused to the N-terminus of aa 1-176 from HA2, aa positions of HA1 and HA2 are based on H3 numbering) (SEQ ID NO:40) fused to the N-terminus of the aa linker described in EXAMPLE 1C (SEQ ID NO:44). The fusion protein of SEQ ID NO 40 fused to the N-terminus of SEQ ID NO:44 was termed ecHA-Uruguay. ecHA-Uruguay was produced and purified as described in EXAMPLE 1D.

### Example 3

### Cloning, Expression and Purification of ecHA from Influenza A H5N1 Strains A/Viet Nam/1203/2004, A/Indonesia/5/2005 and A/Egypt/2321-NAMRU3/2007

DNAs encoding amino acids 11-329 (HA1) followed by amino acid 1-176 (HA2) [HA amino acid positions are based on H3 numbering] from A/Viet Nam/1203/2004 (H5N1) (NCBI accession number ABP51977.1), A/Indonesia/5/2005 (H5N1) (NCBI accession number ABWO6108.1) and (A/Egypt/2321-NAMRU3/2007 (H5N1)) strain (NCBI accession number ABP96850.1) flanked at the 3′ end by a BamHI restriction site and at the 5′ end by an AscI restriction site were optimized for expression in insect cells and produced by gene synthesis (Geneart, Regensburg, Germany). The resulting DNA fragments will be digested with BamHI and AscI (SEQ ID NO:36, 37, 38) and cloned into BamHI-AscI digested expression vector pFastBac1_GP67_HA_PR8 resulting in plasmids pFastBac1_GP67_HA_A/Viet Nam/1203/2004 shortly termed pFastBac1_GP67_HA_A_Viet Nam, pFastBac1_GP67_HA_A/Indonesia/5/2005 termed pFastBac1_GP67_HA_A_Indonesia and pFastBac1_GP67_HA_A/Egypt/2321-NAMRU3/2007 shortly termed pFastBac1_GP67_HA_A_Egypt. This plasmid will encode fusion proteins consisting of the N-terminus containing HA0 from the respective viral strains (ecHA_A_Viet Nam. SEQ ID NO:41, ecHA_A_Indonesia SEQ ID NO:42 and ecHA_A_Egypt SEQ ID NO 43) composed of aa 11-329 from HA1 fused to the N-terminus of aa 1-176 from HA2 (aa positions of HA1 and HA2 are based on H3 numbering) fused to the N-terminus of the aa linker described in EXAMPLE 1C (SEQ ID NO:44). The respective fusion proteins with SEQ ID 44 will be termed ecHA-Vietnam. ecHA-Indonesia and ecHA-Egypt respectively. These proteins will be produced and purified as described in EXAMPLE 1D.

### Example 4

### Cloning, Expression and Purification of ecHA from Influenza A H1N1 Strains A/Brisbane/59/2007 and A/California/04/09

DNAs encoding amino acids 11-329 (HA1) followed by amino acid 1-176 (HA2) [HA amino acid positions are based on H3 numbering] from A/Brisbane/59/2007 (NCBI accession number ACA28844.1) and A/California/04/09 (NCBI accession number ACP41105.1) flanked at the 3′ end by a BamHI restriction site and at the 5′ end by a AscI restriction site will be optimized for expression in insect cells and produced by gene synthesis (Geneart, Regensburg, Germany). The resulting DNA fragment will be digested with BamHI and AscI and cloned into BamHI-AscI digested expression vector pFastBac1_GP67_HA_PR8 resulting in plasmids pFastBac1_GP67_A/Brisbane/59/2007 shortly termed pFastBac1_GP67_HA_A_Brisbane and pFastBac1_GP67_A_California_{—}04_{—}09 shortly termed pFastBac1_GP67_HA_A_California. These plasmids will encode fusion proteins consisting of the N-terminus containing HA0 from the respective viral strains (ecHA A/Brisbane/59/2007 ACA28844.1, SEQ ID NO:73 and ecHA A_California/04/2009 ACP41105.1, SEQ ID NO:74) composed of aa 11-329 from HA1 fused to the N-terminus of aa 1-176 from HA2 (aa positions of HA1 and HA2 are based on H3 numbering) fused to the N-terminus of the aa linker described in EXAMPLE 1D (SEQ ID NO:44). The respective fusion proteins with SEQ ID 44 will be termed ecHA-Brisbane and ecHA-California respectively. These proteins will be produced and purified as described in EXAMPLE 1C.

### Example 5

### Coupling of ecHA-PR8 (H1N1) to Qβ and AP205 Virus-Like Particles

A solution containing 1 mg/ml of the purified ecHA-PR8 protein from EXAMPLE 1 (SEQ ID NO:39 genetically fused to the N-terminus of SEQ ID NO:44) in PBS pH 7.2 was incubated for 5 min at room temperature with a 3 fold molar excess of TCEP for reduction of the C-terminal cysteine residue. A solution of 4 ml of 1 mg/ml Qβ VLPs protein in 20 mM HEPES pH 7.2 was reacted for 30 min at room temperature with 85.2 μl of a SMPH solution (50 mM in DMSO). The reaction solution was dialyzed at 4° C. against two 4 l changes of 20 mM HEPES pH 7.2 over 12 and 2 hours respectively. 1 ml of the derivatized and dialyzed Qβ solution was mixed with 3700, 1850 or 925 μl of the TCEP treated ecHA-PR8 [1 mg/ml] and incubated for 4 h at room temperature for chemical cross linking resulting in the vaccine batches Qβ-ecHA(PR8)-1, Qβ-ecHA(PR8)-2 or Qβ-ecHA(PR8)-3 respectively. Uncoupled protein was removed by size exclusion chromatography using a Sepharose CL4B column. Coupled products were analyzed on a 4-12% Bis-Tris-polyacrylamide gel under reducing conditions. Coomassie staining of the gels reveled several bands of increased molecular weight with respect to the Qβ monomer and the ecHA-PR8 monomer, clearly demonstrating the successful cross-linking of the ecHA-PR8 protein to Qβ VLPs. Densitometric quantification of the coupling bands revealed the following coupling densities for the different vaccine batches: Qβ-ecHA(PR8)-1: 40 ecHA/VLP, Qβ-ecHA(PR8)-2: 29 ecHA/VLP and Qβ-ecHA(PR8)-3:17 ecHA/VLP. For the coupling to AP205 VLPs a solution of 5 ml of 1 mg/ml AP205 VLPs in 20 mM HEPES pH 7.2 was reacted for 90 min at room temperature with 106.5 μl of a SMPH solution (50 mM in DMSO). The reaction solution was dialyzed at 4° C. against three 5 l changes of 20 mM HEPES pH 7.2 over 12, 2 and 2 hours respectively. 2 ml of the derivatized and dialyzed AP205 solution was mixed with 5500 μl of the TCEP treated ecHA-PR8 (H1N1) and incubated 4 h at room temperature for chemical cross linking, resulting in AP205-ecHA(PR8). Uncoupled protein was removed by size exclusion chromatography using a Sepharose CL4B column. Coupled products were analyzed on a 4-12% Bis-Tris-polyacrylamide gel under reducing conditions. The Coomassie stained gel revealed several bands of increased molecular weight with respect to the VLP monomer and the ecHA-PR8 monomer, clearly demonstrating the successful cross-linking of the ecHA-PR8 protein to AP205 VLPs. Densitometric quantification of the coupling bands revealed a coupling density of 30 ecHA/VLP.

### Example 6

### ELISA

For the determination of HA specific antibody titers, ELISA plates were coated either with ecHA-PR8 obtained in EXAMPLE 1, ecHA-Uruguay obtained in EXAMPLE 2, or recombinant influenza HA proteins (rHA) obtained from Protein Sciences (rHA_A/Brisbane/59/2007, rHA_A/Vietnam/1203/2004, rHA_A/Indonesia/05/2005, rHA_A/California/04/2009, rHA B/Florida/04/2006) or alternatively the ELISA plates will be coated with the ecHA proteins obtained in EXAMPLE 3 and EXAMPLE 4 at a concentration of 1 μg/ml or Qβ or AP205 VLPs at a concentration of 10 μg/ml. The plates were blocked and then incubated with serial dilutions of mouse sera. Bound antibodies were detected with enzymatically labeled anti-mouse IgG, anti-mouse IgG1 or anti-mouse IgG2a antibodies. Total IgG antibody titers were determined as the reciprocals of the dilutions required to reach 50% of the optical density (OD450 nm) measured at saturation. For IgG1 and IgG2a endpoint titers were calculated. Mean antibody titers are shown.

### Example 7

### Determination of Hemagglutination Inhibition Titers of Influenza Virus PR8

Sera of mice were tested for their ability to inhibit the agglutination of chicken red blood cells by influenza virus PR8. To inactivate non-specific inhibitors, sera were first treated with receptor destroying enzyme (RDE, Seiken, Japan). Briefly, three parts RDE was added to one part sera and incubated overnight at 37° C. RDE was inactivated by incubation at 56° C. for 30 min. Depending on the dilution of the sera, 0 to 6 parts of PBS were added for a final 1:4 to 1:10 dilution of the sera. RDE-treated sera were serially diluted two-fold in v-bottom microtiter plates. An equal volume of influenza PR8 virus, adjusted to 8 HAU/50 ul, was added to each well. The plates were covered and incubated at room temperature for 30 min followed by the addition of 1% chicken erythrocytes in PBS. The plates were mixed by agitation, covered, and the RBCs were allowed to settle for 1 h at room temperature. The HAI titer was determined as the reciprocal of the dilution of the last row which contained non-agglutinated RBC. To determine the HAI titers against other influenza virus strains the respective virus strain is used (instead of influenza A/PR/8/34) for agglutination of RBCs. For these other influenza strains RBCs from different species (e.g. turkey or horse) may have to be used for agglutination.

### Example 8

### Murine Influenza Model

The following influenza A viruses were used in the different studies: A/PR/8/34 (H1N1), A/FM/1/47 (H1N1), A/Aichi/2/68 (X31) (H3N2) and A/WSN/33 (H1N1). To determine the lethal dose of each virus, mice were administered serial dilutions of virus (2×50 μl) via the nose under light anesthesia with isofuran. Body weight and body temperature of infected mice were monitored for at least 20 days after infection. Mice, which had lost more than 30% of their initial body weight or had a body temperature equal to or lower than 30° C. were euthanized. LD50 titers were calculated for each virus strain according to the method of Reed and Munch (Reed L J et al. 1938. Am. J. Hyg. 27, 493-497). To determine the efficacy of the different vaccines, mice were immunized with the indicated compounds and challenged with a lethal dose of homologous or heterologous influenza virus (4LD50 or 10LD50) as indicated in the respective examples and monitored as described above. Mice that had lost more than 30% of their initial body weight or had a body temperature equal to or lower than 30° C. were euthanized. The % surviving animals 20 days post infection (p.i.) for each treatment group is indicated in the respective examples.

### Example 9

### Qβ-ecHA(PR8) and AP205-ecHA(PR8) Vaccines Protection from a Lethal Homologous Influenza Challenge

Three female balb/c mice per group were immunized s.c. on day 0 with 50, 5 or 0.5 of Qβ-ecHA(PR8)-1, Qβ-ecHA(PR8)-2 or Qβ-ecHA(PR8)-3 (obtained in EXAMPLE 5) or 45 or 4.5 μg of ecHA(PR8) (obtained in EXAMPLE 1) or 50 μg of Qβ VLPs formulated in 200 μl PBS. Sera were collected by retro-orbital bleeding on day 20 and analyzed using ecHA (PR8)-specific ELISA or hemagglutination inhibition (HAI) assay as described in EXAMPLEs 6 and 7. On day 21 all mice were challenged with 4LD50 of mouse adapted influenza virus A/PR/8/34 and monitored for 20 days for survival as described in EXAMPLE 8. The results of this experiment are shown in Table 1. As shown in Table 1 all animals that had been immunized with any of the three Qβ-ecHA(PR8) conjugate at every concentration tested survived the lethal challenge whereas all animals that had been immunized with the carrier alone (Qβ) died. Only partial protection was observed in animals that had received ecHA(PR8) alone at both concentrations tested. Likewise, ecHA-PR8 specific titers and HAI titers were significantly increased in all animals that had received Qβ-ecHA(PR8) compared to the animals that had been immunized with ecHA(PR8) alone. The induced HAI titers were proportional to the anti-ecHA(PR8) antibody ELISA titers suggesting that the induced antibodies recognize native HA on the virus. These results demonstrates that coupling of ecHA-PR8 to Qβ VLPs, even with low coupling density is strongly enhancing the immunogenicity of ecHA-PR8, whereas the immune response of the Qβ VLPs is strongly reduced when antigens are coupled to the VLP which minimizes the risk of carrier induced epitopic suppression. Moreover, a single immunization with 0.5 μg of Qβ-ecHA(PR8) with a low coupling density (17 HA/VLP) was able to fully protect mice from a lethal challenge with the homologous influenza virus A/PR8/34.

TABLE 1

| | | Anti-ecHA-PR8- | Anti-Qβ-IgG | HAI titer | Survival in % |
|---|---|---|---|---|---|
| Antigen | Amount [μg] | IgG d20 | d20 | d20 | 20 d p.i. |
| Qβ-ecHA(PR8)-1 | 50 | 10′165 | 2′218 | 52 | 100 |
| | 5 | 4′549 | 505 | 17 | 100 |
| | 0.5 | 1′569 | 85 | 8 | 100 |
| Qβ-ecHA(PR8)-2 | 50 | 16′607 | 4′447 | 75 | 100 |
| | 5 | 8′589 | 662 | 43 | 100 |
| | 0.5 | 3′293 | 117 | 11 | 100 |
| Qβ-ecHA(PR8)-3 | 50 | 23′487 | 6′073 | 192 | 100 |
| | 5 | 4′905 | 1′241 | 8 | 100 |
| | 0.5 | 4′390 | 696 | 11 | 100 |
| ecHA(PR8) | 45 | 186 | 40 | 0 | 66 |
| | 4.5 | 430 | 182 | 0 | 33 |
| Qβ | 50 | 1 | 73′483 | 0 | 0 |

### Example 10

### Dose Titration of Qβ-ecHA(PR8) in Lethal Challenge Studies

To further determine the protective potential of the vaccine, five female balb/c mice per group were immunized with 5, 1, 0.2, 0.04, 0.008 μg of Qβ-ecHA(PR8)-1 (obtained in EXAMPLE 5) or 15 μg of total protein of ecHA(PR8) (obtained in EXAMPLE 1) or as a negative control with 50 μg of Qβ VLPs. All compounds were formulated in 200 μl PBS and injected subcutaneously on day 0. Mice were bled retro-orbitally on day 21 and sera were analyzed using ecHA(PR8)-specific ELISA or HA1 assay. On day 63 all mice were challenged with 4LD50 of mouse adapted influenza virus A/PR/8/34 and monitored for 20 days for survival (as described in EXAMPLE 8). The results of this experiment are shown in Table 2. As shown in Table 2, a single injection of 0.008 μg of Qβ-ecHA(PR8)-1 induced a higher anti-HA(PR8)-IgG and HAI titer than 15 μg of ecHA(PR8). Moreover similar protection against a lethal challenge with mouse adapted influenza A/PR/8/34 was observed with 0.008 μg of Qβ-ecHA(PR8)-1 than with 15 μg of ecHA(PR8). This demonstrates that coupling of ecHA-PR8 to Qβ VLPs allows about a thousand fold dose sparing of ecHA-PR8 antigen, since 0.008 μg of Qβ-ecHA(PR8)-1 induced a similar response and protection than 15 μg of ecHA(PR8), which is the standard dose of influenza HA included into commercial TIV influenza vaccines.

TABLE 2

| | | | | Survival |
|---|---|---|---|---|
| | Amount | Anti-ecHA-PR8-IgG | HAI titer | [%] |
| Antigen | [μg] | d21 | d21 | 20 d p.i. |
| Qβ-ecHA(PR8)-1 | 5 | 6′460 | 27 | 100 |
| | 1 | 2′223 | 16 | 100 |
| | 0.2 | 1′121 | 6 | 100 |
| | 0.04 | 184 | 2 | 100 |
| | 0.008 | 266 | 6 | 100 |
| ecHA(PR8 | 15 | 41 | 3 | 100 |
| Qβ | 50 | 0 | 4 | 0 |

### Example 11

### Dose Titration of Qβ-ecHA(PR8) and AP205-ecHA(PR8) in Lethal Challenge Studies

Next the protective potential of a HA vaccine based on another bacteriophage carrier was assessed. To this end, four female balb/c mice per group were immunized with 15, 3, 0.6, 0.12, 0.024, 0.0046 μg of AP205-ecHA(PR8) obtained in EXAMPLE 5 or 15 μg of Qβ-ecHA(PR8)-1 obtained in EXAMPLE 5 or 15 μg of ecHA(PR8) obtained in EXAMPLE 1 or 50 μg Qβ VLPs. All compounds were formulated in 200 μl PBS and injected s.c. on day 0. Mice were bled retro-orbitally on day 21 and sera were analyzed using ecHA(PR8)-specific ELISA or HA1 assay as described in EXAMPLES 6 and 7. On day 27 all mice were challenged with 4LD50 of mouse adapted influenza virus A/PR/8/34 and monitored for 20 days for survival as described in EXAMPLE 8. The results of this experiment are shown in Table 3. As shown in Table 3, coupling of ecHA-PR8 to AP205 VLPs strongly enhanced the immunogenicity of ecHA-PR8 and allowed an approximately 625 fold dose sparing of ecHA-PR8 antigen, since 0.024 μg of AP205-ecHA (PR8) induced similar anti-HA(PR8)-IgG titers and HAI titers than 15 μg of HA(PR8), which is the standard dose of influenza HA included into commercial TIV influenza vaccines. Moreover, a single dose of 0.024 μg of AP205-ecHA completely protected mice from a lethal influenza challenge. Interestingly the response induced by ecHA coupled to AP205 VLPs induced higher IgG2a than IgG1 titers whereas ecHA(PR8) alone induced higher IgG1 than IgG2a titer, suggesting that coupling to VLPs induces a shift from a TH2 to a TH1 immune response.

TABLE 3

| | | Anti-ecHA-PR8-IgG | | | HAI | Survival |
|---|---|---|---|---|---|---|
| | Amount | IgG | IgG1 | IgG2a | titer | in [%] |
| Antigen | [μg] | d 21 | d 21 | d 21 | d 21 | 20 d p.i. |
| AP205- | 15 | 7'246 | 3'107 | 5'362 | 30 | 100 |
| ecHA(PR8) | 3 | 2'864 | n.d. | n.d | 38 | 100 |
| | 0.6 | 1'291 | | | 41 | 100 |
| | 0.12 | 1'474 | | | 18 | 100 |
| | 0.024 | 695 | | | 17 | 100 |
| | 0.0046 | 173 | | | 10 | 75 |
| ecHA(PR8) | 15 | 658 | 3'023 | 54 | 5 | 100 |
| AP205 | 50 | 0 | n.d. | n.d. | 3 | 0 |

### Example 12

### Induction of Cross-Protection with Qβ-ecHA(PR8) and AP205-ecHA(PR8) in Lethal Influenza Challenge Experiments

To further determine the protective potential of the HA vaccines, six female balb/c mice per experimental group were immunized with 15 μg of Qβ-ecHA(PR8)-1 obtained in EXAMPLE 5 or 15 μg AP205-ecHA(PR8) obtained in EXAMPLE 5 or 15 μg of ecHA(PR8) obtained in EXAMPLE 1 or 15 μg of Qβ or AP205. All proteins were formulated in 200 μl PBS and injected subcutaneously either two times (on day 0 and day 21) or only once on day 21 (see also Table 4 for more details). Mice were bled retro-orbitally on day 35 and sera were analyzed by ELISA or HAI assay as described in EXAMPLE 6 and 7. On day 39 the respective groups were challenged with 10LD50 of A/PR/8/34 (H1N1), 10LD50 A/WSN/33 (H1N1), 10LD50 A/FM/1/47 (H1N1) or 10LD50 A/Aichi/2/68 (X31) (H3N2) as outlined in Table 4. Mice were then monitored for survival as described in EXAMPLE 8. The results of this experiment are shown in Table 4. As shown in Table 4, immunization of mice with ecHA(PR8) coupled to Qβ or AP205 is inducing protection against infection with a high lethal dose (10LD50) of the homologous influenza A/PR8/34 and the heterologous A/WSN/33 virus after a single injection. In contrast a single immunization with ecHA(PR8) failed to protect against a heterologous challenge with A/WSN/33 and only partly protected against a homologous challenge with A/PR/8/34. For full protection against a homologous or heterologous challenge with A/WSN/33 a second immunization was required with ecHA(PR8). Likewise ecHA(PR8) coupled to Qβ or AP205 showed a clearly improved cross-protection after one and two immunizations compared to ecHA(PR8) when the mice were challenged with the A/FM/1/47-MA (H1N1) strain since neither 1 nor 2 injections with ecHA(PR8) alone was able to fully protect the mice from a lethal challenge. Immunization of mice with ecHA(PR8) alone or coupled to Qβ or AP205 induced some degree of cross-protection against a lethal infection (10LD50) of mice with the H3N1 influenza strain A/Aichi/2/68 (X31) virus. The level of cross-protection did not correlate to anti-ecHA(PR8) IgG antibody titers, indicating that ecHA(PR8)-specific IgG antibodies might not be responsible for cross-protection in this case suggesting a different mechanism for cross-protection being in place in these experimental groups. Taken together these experiments further emphasize that the coupling of the ecHA to the surface of bacteriophage (AP205 or Qβ) VLPs clearly enhances its immunogenicity and improves the protective response induced against HA. This is particularly highlighted by the fact the bacteriophage-ecHA vaccines are able to fully protect against the challenge with a heterologous virus whilst the ecHA alone is not.

TABLE 4

| | No of | Anti-ecHA- | Anti-Qβ- | Anti-AP205- | Challenge | Survival [%] |
|---|---|---|---|---|---|---|
| Antigen | immunizations | PR8-IgG d 35 | IgG d 35 | IgG d 35 | virus strain | 20 d p.i. |
| Qβ-ecHA(PR8)-1 | 2 | 17'300 | 942 | n.d. | A/PR8/34 | 100 |
| AP205-ecHA(PR8) | | 15'561 | n.d. | 457 | | 100 |
| ecHA(PR8) | | 27'345 | n.d. | n.d. | | 100 |
| Qβ | | n.d. | 156'796 | n.d. | | 0 |
| AP205 | | n.d. | n.d. | 23'770 | | 0 |
| Qβ-ecHA(PR8)-1 | 1 | 835 | 135 | n.d. | | 100 |
| AP205-ecHA(PR8) | | 815 | n.d. | 236 | | 100 |
| ecHA(PR8) | | 37 | n.d. | n.d. | | 33 |
| Qβ | | n.d. | 156'796 | n.d. | | 0 |
| AP205 | | n.d. | n.d. | 53'813 | | 0 |
| Qβ-ecHA(PR8)-1 | 2 | 16'361 | 1'246 | n.d. | A/WSN/33 | 100 |
| AP205-ecHA(PR8) | | 20'954 | n.d. | 511 | | 100 |
| ecHA(PR8) | | 35'011 | n.d. | n.d. | | 100 |
| Qβ | | n.d. | 179'208 | n.d. | | 0 |
| AP205 | | n.d. | n.d. | 52'385 | | 0 |
| Qβ-ecHA(PR8)-1 | 1 | 3'375 | 239 | n.d. | | 83 |
| AP205-ecHA(PR8) | | 852 | n.d. | 128 | | 100 |
| ecHA(PR8) | | 20 | n.d. | n.d. | | 0 |
| Qβ | | n.d. | 120'294 | n.d. | | 0 |
| AP205 | | n.d. | n.d. | 23'359 | | 0 |
| Qβ-ecHA(PR8)-1 | 2 | 45'425 | 3'270 | n.d. | A/FM/1/47- | 100 |
| AP205-ecHA(PR8) | | 11'297 | n.d. | 700 | MA | 100 |
| ecHA(PR8) | | 25'843 | n.d. | n.d. | | 66 |
| Qβ | | n.d. | 141'008 | n.d. | | 0 |
| AP205 | | n.d. | n.d. | 46'341 | | 0 |
| Qβ-ecHA(PR8)-1 | 1 | 4'757 | 505 | n.d. | | 100 |
| AP205-ecHA(PR8) | | 712 | n.d. | 203 | | 100 |
| ecHA(PR8) | | 27 | n.d. | n.d. | | 33 |
| Qβ | | n.d. | 170'457 | n.d. | | 0 |
| AP205 | | n.d. | n.d. | 21'695 | | 0 |
| Qβ-ecHA(PR8)-1 | 2 | 40'141 | 2'613 | n.d. | A/Aichi/2/68 | 17 |
| AP205-ecHA(PR8) | | 10'467 | n.d. | 612 | (X31) | 50 |
| ecHA(PR8) | | 20 | n.d. | n.d. | | 17 |
| Qβ | | n.d. | 170'457 | n.d. | | 0 |
| AP205 | | n.d. | n.d. | 65'849 | | 0 |
| Qβ-ecHA(PR8)-1 | 1 | 5'018 | 405 | n.d. | | 50 |
| AP205-ecHA(PR8) | | 998 | n.d. | 209 | | 17 |
| ecHA(PR8) | | 14'732 | n.d. | n.d. | | 17 |
| Qβ | | n.d. | 141'008 | n.d. | | 0 |
| AP205 | | n.d. | n.d. | 27'520 | | 0 |

### Example 13

### Production and Testing of a Vaccine Against an Influenza H3N2 Strain

ecHA-A-Uruguay obtained from EXAMPLE 2 was coupled to Qβ VLPs as described in EXAMPLE 5. The Immunogenicity of this vaccine was tested in mice. Briefly, four female balb/c mice per group were immunized with 15, 3, 0.6, 0.12, 0.024, 0.0046 μg of Qβ-ecHA(Uruguay) or 15 μg of ecHA(Uruguay) obtained in EXAMPLE 2 or 50 μg Qβ VLPs. All compounds were formulated in 200 μl PBS and injected s.c. on day 0. Mice were bled retro-orbitally on day 21 and sera were analyzed using ecHA-Uruguay-specific ELISA. The results are summarized in Table 5. As shown in Table 5 coupling of ecHA-Uruguay to Qβ VLPs dramatically increased its immunogenicity since 0.0046 μg of the vaccine induced a higher ecHA specific ELISA titer than 15 μg of the ecHA(Uruguay) alone.

TABLE 5

| Antigen | Amount [μg] | IgG d21 |
|---|---|---|
| Qβ-ecHA(Uruguay) | 15 | 6879 |
| | 3 | 3023 |
| | 0.6 | 1533 |
| | 0.12 | 1060 |
| | 0.024 | 790 |
| | 0.0046 | 1832 |
| ecHA(Uruguay) | 15 | 478 |
| Qβ | 0 | 20 |

### Example 14

### Production and Testing of Vaccines Against Influenza H5N1 and H1N1 Strains

ecHA-Vietnam, ecHA-Indonesia, ecHA-Egypt, ecHA-Brisbane and ecHA-California obtained from EXAMPLE 3 and 4 will be coupled to Qβ and AP205 VLPs as described in Example 5. The efficacy of these vaccines will be tested in a mouse model for influenza infection as described in EXAMPLE 8. ELISA antibody titers and HAI titers in sera from immunized mice will be determined as described in EXAMPLES 6 and 7 with the appropriate coating reagent and virus strain used for the hemagglutination test. In addition dose titration experiments, where the immunized animals will be challenged with a homologous virus similar to the experiment described in EXAMPLE 10 will be performed. Moreover to evaluate the protective potential further, cross protection experiments in which the animals will be either challenged with the homologous influenza virus or a heterologous influenza virus strain will be performed similar to the experiment described in EXAMPLE 12.

### Example 15

### In Vitro Neutralization of Influenza Virus by Sera from Vaccinated Animals

Sera of immunized mice obtained in EXAMPLES 9-14 and 26-33 will used in in vitro neutralization assays. Briefly, homologous and heterologous influenza viruses will be incubated with serial dilutions of the respective sera and the ability to inhibit the MDCK cells with the respective influenza virus will be determined. The virus neutralization titers will be defined as the reciprocal of the highest serum dilution capable of completely inhibiting 200 TCID50 of the respective influenza virus from infecting MDCK monolayers in a microtiter plate. Infection will be measured by an ELISA which determines intracellularly produced viral NP protein.

### Example 16

### Cloning, Expression, Purification and Refolding of Different Fragments of the Globular Domain of HA (gdHA) of Mouse Adapted Influenza A/PR/8/34 (H1N1) Virus

A) Generation of pET-42T(+)

pET-42T(+) is a derivative of pET-42a(+) (Novagen), where a 6×His-tag and the aa linker (GGC) followed by a stop codon was introduced after the multiple cloning site for expression of fusion-proteins with a C-terminus encoding the aa sequence of SEQ ID NO:91. In a first step the intermediate vector pET-42S(+) was constructed by ligating the annealed pair of oligo 42-1 (SEQ ID NO:45) and oligo 42-2 (SEQ ID NO:46) into the NdeI-AvrII digested pET-42a(+) plasmid to obtain pET-42S(+). In a second step the annealed pair of oligo 42T-1 (SEQ ID NO:47) and oligo 42T-2 (SEQ ID NO:48) was ligated into the XhoI-AvrII digested pET-42S (+) plasmid to obtain the vector pET-42T (+) (SEQ ID NO:60). The resulting plasmid has NdeI, EcoRV, EcoRI, HindIII, PstI, PvuII, XhoI, XcmI, AvrII restriction sites in its multiple cloning site.

B) Generation of Constructs gdHA_PR8_{—}42_{—}310, gdHA_PR8_{—}46_{—}310, gdHA_PR8_{—}57_{—}276, gdHA_PR8_{—}54a_{—}276, gdHA_PR8_{—}54a_{—}270, gdHA_PR8_{—}57_{—}270

Fragments of the ectodomain of HA (gdHA) of mouse adapted influenza A A/PR/8/34 (H1N1) virus (prototype H1 HA fragments) were designed based on the protein structure (PDB 1RVX) of prototype human (1934—human) H1 influenza virus A/Puerto Rico/8/34 HA described in Gamblin S J et al., Science, 2004 303:1838-42. Based on aa sequence alignment of mouse adapted A/PR/8/34 (SEQ ID NO:39, obtained in EXAMPLE 1B) with the prototype human (1934—human) H1 influenza virus A/Puerto Rico/8/34 HA (Gamblin S J et al., Science, 2004 303:1838-42) the nucleotide sequence encoding amino acids 36-311 (HA1) corresponding to amino-acids 42-310 (HA1) based on H3 numbering (Stevens J, Science 2004 303, 1866-1870) flanked by a NdeI restriction site at the N-terminus and by a XhoI restriction site at the C-terminus was optimized for expression in E. coli and produced by gene synthesis (Geneart, Regensburg, Germany). The optimize nucleotide sequence was digested with NdeI and XhoI and cloned into NdeI-XhoI sites of pET-42T(+) resulting in plasmid pET42T_HA1_PR8_{—}42_{—}310 (SEQ ID NO:61). This vector was used to generate different shorter fragments by PCR as outlined in Table 6. Briefly, PCR reactions were performed with the indicated primers on pET42T_HA1_PR8_{—}42_{—}310 and the resulting products were digested with NdeI and XhoI and cloned into NdeI-XhoI sites of pET-42T(+) resulting in the constructs indicated in the last column of Table 6. These plasmids encode fusion proteins consisting of an N-terminus composed of the aa sequences aa42-310 (SEQ ID NO:67), aa 46-310 (SEQ ID NO:68), aa57-276 (SEQ ID NO:69), aa54a-276 (SEQ ID NO:70), aa54a-270 (SEQ ID NO:71), and aa57-270 (SEQ ID NO:72) of the ectodomain of mouse adapted influenza virus A/PR/8/34 (SEQ ID NO:39) genetically fused to the N-terminus of SEQ ID NO:91. Amino acid positions are according to H3 numbering derived from Stevens J. et al, Science 2004 303, 1866-1870). The resulting proteins were named gdHA_PR8_{—}42_{—}310, gdHA_PR8_{—}46_{—}310, gdHA_PR8_{—}57_{—}276, gdHA_PR8_{—}54a_{—}276, gdHA_PR8_{—}54a_{—}270, gdHA_PR8_{—}57_{—}270, respectively.

TABLE 6

| | | Construct Name |
|---|---|---|
| Oligo 1 | Oligo 2 | (NdeI/XhoI fragment) |
| JA35 | JA40 | pET42T_HA1_PR8_46_310 |
| (SEQ ID NO: 51) | (SEQ ID NO: 52) | (SEQ ID NO: 62) |
| JA37 | JA39 | pET42T_HA1_PR8_57_276 |
| (SEQ ID NO: 53) | (SEQ ID NO: 54) | (SEQ ID NO: 63) |
| JA36 | JA39 | pET42T_HA1_PR8_54a_276 |
| (SEQ ID NO: 55) | (SEQ ID NO: 56) | (SEQ ID NO: 64) |
| JA36 | JA38 | pET42T_HA1_PR8_54a_270 |
| (SEQ ID NO: 55) | (SEQ ID NO: 58) | (SEQ ID NO: 65) |
| JA37 | JA38 | pET42T_HA1_PR8_57_270 |
| (SEQ ID NO: 53) | (SEQ ID NO: 58) | (SEQ ID NO: 66) |

C) Expression, Purification and Refolding of gdHA Constructs

For expression, Escherichia coli BL21 cells harboring either plasmid were grown at 37° C. to an OD at 600 nm of 1.0 and then induced by addition of isopropyl-β-D-thiogalactopyranoside at a concentration of 1 mM. Bacteria were grown for 4 more hours at 37° C., harvested by centrifugation and resuspended in 5 ml lysis buffer (50 mM Na₂HPO₄, 300 mM NaCl, 10 mM Imidazole, pH 8.0) per gram wet weight and cells were lysed by 30 min incubation with 1 mg/ml lysozyme. Cells were then disrupted by sonication and cellular DNA was digested by 15 min incubation on ice with 5 μg/ml DNAse I. Inclusion bodies (IB) were harvested by centrifugation (10,000×g, 4° C., 30 min), purified using B-PER I reagent (Pierce) and solubilized in IB solubilisation buffer (8 M urea, 50 mM Tris-Cl pH 8.0, 50 mM Dithiothreitol) to a concentration of 0.5 mg/ml. Refolding of proteins was performed by dialysis against refolding buffer 2 (2 M urea, 50 mM NaH₂PO₄, 0.5 M Arginine, 10% Glycerole (v/v), 5 mM Glutathion reduced, 0.5 mM Glutathion oxidized, pH 8.5), followed by dialysis against refolding buffer 3 (50 mM NaH₂PO₄, 0.5 M Arginine, 10% Glycerole (v/v), 5 mM Glutathion reduced, 0.5 mM Glutathion oxidized, pH 8.5), followed by dialysis against refolding buffer 4 (20 mM Sodium-Phosphate, 10% Glycerole (v/v), pH 7.2. Refolded proteins were stored at −80° C. until further use.

### Example 17

### Design and Numbering of Fragments of the Ectodomain of Influenza A Subtypes H1, H2, H3, H4, H5, H6, H7, H8, H9, H10, H11, H12, H13, H14, H15, H16 HA of Naturally Occurring Influenza A Viruses and Influenza B Viruses

Based on the structure of the H1 HA of the human 1934-H1N1 influenza A strain (pdb 1RVX) (Gamblin S J et al, Science, 2004 303, 1838-1842) influenza A H1 HA prototype fragments were designed as described in EXAMPLE 16B. The influenza A H1 HA prototype fragments was structurally aligned to the structure of a influenza HA of the H3 subtype (human 1968-H3N2 influenza A strain (pdb 1E08), Wilson I A et al, Nature (1981) 289, 366-373), to the structure of an influenza HA of H5 subtype namely human 2004-H5N1 influenza A strain (pdb 2 FK0) (Stevens J et al, Science (2006) 312, 404-410) and human influenza B virus B/Hong Kong/8/73 (pdb 3BT6) (Wang Q et al, J. Virol (2008) 3011-3020) to design influenza A H3 prototype, influenza A H5 prototype HA fragments and influenza B prototype HA fragments with similar structures as the influenza A H1 HA prototype fragments. Numbering of the fragments was based on the human 1968-H3N2 influenza A strain (pdb 1E08) (Wilson I A et al, Nature (1981) 289, 366-373). Influenza A H1, H3 and H5 fragments of naturally occurring influenza viruses were designed by aa alignment with the prototype HA fragments of the corresponding subtypes of influenza A virus strains. Influenza A H6, H13, H11, H16 HA fragments of naturally occurring influenza A viruses will be designed by aa alignment or structural modeling and structural alignment with the prototype H1 HA fragments, influenza A H4, H7, H10, H14, H15 HA fragments of naturally occurring influenza viruses will be designed by aa alignment or structural modeling and structural alignment with the prototype H3 HA fragments, influenza A H2, H8, H9, H12 HA fragments of naturally occurring influenza viruses will be designed by aa alignment or structural modeling and structural alignment with the prototype H5 HA fragments and numbered according to H3 numbering (Wilson I A et al, Nature (1981) 289, 366-373). Model building will be carried out using the program SWISS-MODEL.

### Example 18

### Cloning, Expression, Purification and Refolding of gdHA Fragments from Influenza A/California/04/2009

The cDNA of HA0 of influenza A (A/California/04/09) (H1N1)) strain (NCBI accession number ACP41105.1) encoding amino acids 42-310 (based on H3 numbering) flanked at the 3′ end by a NdeI restriction site and at the 5′ end by a XhoI restriction site was optimized for expression in E. coli and produced by gene synthesis by Geneart, Regensburg, Germany. The optimized nucleotide sequence was digested with NdeI and XhoI (SEQ ID NO:77) and cloned into the NdeI-XhoI sites of pET-42T(+) resulting in plasmid pET42T_HA1_AC0409_{—}42_{—}310. This plasmid encodes aa42-310 of the ectodomain of influenza virus A/California/04/09 (SEQ ID NO:84) fused to the N-terminus of SEQ ID NO:91 and was termed gdHA_AC0409_{—}42_{—}310 and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) of the globular domain of A/California/04/2009, flanked by NdeI and XhoI sites, will be amplified with appropriate oligonucleotides and cloned into pET-42T(+) in analogy to Example 16B. These proteins will be purified and refolded as described in EXAMPLE 16C.

### Example 19

### Cloning, Expression, Purification and Refolding gdHA Fragments from Influenza A/Brisbane/59/2007 IVR148 (H1N1)

The cDNA of HA0 of influenza A (A/Brisbane/59/2007) (H1N1)) strain (NCBI accession number ACA28844.1) encoding, based on H3 numbering, amino acids 42-310 flanked at the 3′ end by a NdeI restriction site and at the 5′ end by a XhoI restriction site was optimized for expression in E. coli and produced by gene synthesis by Geneart, Regensburg, Germany. The optimized nucleotide sequence was digested with NdeI and XhoI (SEQ ID NO:78) and cloned into the NdeI-XhoI sites of pET-42T(+) resulting in plasmid pET42T_HA1_AB5907_{—}42_{—}310. This plasmid encodes aa42-310 of the ectodomain of influenza virus A/Brisbane/59/2007 (H1N1) (SEQ ID NO:85) fused to the N-terminus of SEQ ID NO:91 and was termed gdHA_AB5907_{—}42_{—}310 and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) of the globular domain of A/Brisbane/59/2007 IVR148, flanked by NdeI and XhoI sites, will be amplified with appropriate oligonucleotides and cloned into pET-42T(+) in analogy to EXAMPLE 16B. These proteins will be purified and refolded as described in EXAMPLE 16C.

### Example 20

### Cloning, Expression, Purification and Refolding gdHA Fragments from Influenza A/Uruguay/716/2007/NYMC/X/175C(H3N2)

The cDNA of HA0 of influenza A (A/Uruguay/716/2007 X-175 (H3N2)) strain (NCBI accession number ACD47234.1) encoding amino acids 42-310 (based on H3 numbering) flanked at the 3′ end by a NdeI restriction site and at the 5′ end by a XhoI restriction site was optimized for expression in E. coli and produced by gene synthesis by Geneart, Regensburg, Germany. The optimized nucleotide sequence was digested with NdeI and XhoI (SEQ ID NO:79) and cloned into the NdeI-XhoI sites of pET-42T(+) resulting in plasmid pET42T_HA1_AU71607_{—}42_{—}310. This plasmid encodes aa42-310 of the ectodomain of influenza virus A/Uruguay/716/2007 (X-175) H3N2 (SEQ ID NO:86) fused to the N-terminus of SEQ ID NO:91 and was termed gdHA_AU71607_{—}42_{—}310 and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) of the globular domain of A/Uruguay/716/2007/NYMC/X/175C flanked by NdeI and XhoI sites, will be amplified with appropriate oligonucleotides and cloned into pET-42T(+) in analogy to EXAMPLE 16B. These proteins will be purified and refolded as described in EXAMPLE 16C.

### Example 21

### Cloning, Expression, Purification and Refolding gdHA Fragments from Influenza A/Viet Nam/1203/2004 (H5N1)

The cDNA of HA0 of influenza A (A/Viet Nam/1203/2004 (H5N1)) strain (NCBI accession number ABP51977.1) encoding, amino acids 42-310 (based on H3 numbering) flanked at the 3′ end by a NdeI restriction site and at the 5′ end by a XhoI restriction site was optimized for expression in E. coli and produced by gene synthesis by Geneart, Regensburg, Germany. The optimized nucleotide sequence was digested with NdeI and XhoI (SEQ ID NO:81) and cloned into the NdeI-XhoI sites of pET-42T(+) resulting in plasmid pET42T_HA1_AV120304_{—}42_{—}310. This plasmid encodes aa42-310 of the ectodomain of influenza virus A/VietNam/1203/2004 (H5N1) (SEQ ID NO:88) fused to the N-terminus of SEQ ID NO:91 and was termed gdHA_AV120304_{—}42_{—}310 and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) of the globular domain of A/Viet Nam/1203/2004 flanked by NdeI and XhoI sites, will be amplified with appropriate oligonucleotides and cloned into pET-42T(+) in analogy to EXAMPLE 16B. These proteins will be purified and refolded as described in EXAMPLE 16C.

### Example 22

### Cloning, Expression, Purification and Refolding gdHA Fragments from Influenza A/Indonesia/5/2005 (H5N1)

The cDNA of HA0 of influenza A (A/Indonesia/5/2005 (H5N1)) strain (NCBI accession number ABWO6108.1) encoding, amino acids 42-310 (based on H3 numbering) flanked at the 3′ end by a NdeI restriction site and at the 5′ end by a XhoI restriction site was optimized for expression in E. coli and produced by gene synthesis by Geneart, Regensburg, Germany. The optimized nucleotide sequence was digested with NdeI and XhoI (SEQ ID NO:82) and cloned into the NdeI-XhoI sites of pET-42T(+) resulting in plasmid pET42T_HA1_AI505_{—}42_{—}310. This plasmid encodes aa42-310 of the ectodomain of influenza virus A/Indonesia/5/2005 (H5N1) fused to the N-terminus of SEQ ID NO:91 and was termed gdHA_AI505_{—}42_{—}310 (SEQ ID NO:89) and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) of the globular domain of A/Indonesia/5/2005 flanked by NdeI and XhoI sites, will be amplified with appropriate oligonucleotides and cloned into pET-42T(+) in analogy to Example 16B. These proteins will be purified and refolded as described in EXAMPLE 16C.

### Example 23

### Cloning, Expression, Purification and Refolding gdHA Fragments from Influenza Influenza B/Brisbane/3/07

The cDNA of HA0 of influenza B (B/Brisbane/3/2007) strain (accession number ISDN263782) encoding amino acids 42-310 (based on H3 numbering) flanked at the 3′ end by a NdeI restriction site and at the 5′ end by a XhoI restriction site was optimized for expression in E. coli and produced by gene synthesis by Geneart, Regensburg, Germany. The optimized nucleotide sequence was digested with NdeI and XhoI (SEQ ID NO:80) and cloned into the NdeI-XhoI sites of pET-42T(+) resulting in plasmid pET42T_HA1_BB307_{—}42_{—}310. This plasmid encodes aa42-310 of the ectodomain of influenza virus B/Brisbane/3/2007 (SEQ ID NO:87) fused to the N-terminus of SEQ ID NO:91 and was termed gdHA_BB307_{—}42_{—}310 and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) sites of the globular domain of B/Brisbane/3/07 flanked by NdeI and XhoI sites, will be amplified with appropriate oligonucleotides and cloned into pET-42T(+) in analogy to EXAMPLE 16B. These proteins will be purified and refolded as described in EXAMPLE 16C.

### Example 24

### Cloning, Expression, Purification and Refolding gdHA Fragments from Influenza A/California/07/2009 (H1N1)

The cDNA of HA0 of influenza A (A/California/07/09) (H1N1)) strain (NCBI accession number ACR78583) encoding amino acids 42-310 based on H3 numbering flanked at the 3′ end by a XbaI restriction site and at the 5′ end by a HindIII restriction site was optimized for expression in E. coli and produced by gene synthesis by Geneart, Regensburg, Germany. The optimized nucleotide sequence was digested with XbaI-HindIII (SEQ ID NO:83) and cloned into the XbaI-HindIII sites of vector pET-42T(+) resulting in plasmid pET_HA1_AC0709_{—}42_{—}310. This plasmid encodes aa42-310 of the ectodomain of influenza virus A/California/07/09 (H1N1) (SEQ ID NO:90) fused to the N-terminus of aa linker GGCG and was termed gdHA_AC0709_{—}42_{—}310 and was produced, purified and refolded as described in EXAMPLE 16C. Alternatively, pET-42T(+) expression constructs containing shorter fragments (aa 46-310, aa57-276, aa54a-276, aa54a-270 and aa57-270 based on H3 Numbering) of the globular domain of A/California/07/2009 flanked by XbaI and Hind III sites, will be amplified with appropriate oligonucleotides and cloned into pET-42T(+) in analogy to EXAMPLE 16B. These proteins will be purified and refolded as described in EXAMPLE 16C.

### Example 25

### Coupling of Globular Domains of A/PR/A/34 HA to Qβ and AP205 VLPs

A solution of 6 ml of 1 mg/ml Qβ VLPs protein in 20 mM HEPES pH 7.2 was reacted for 30 min at room temperature with 128 μl of a SMPH solution (50 mM in DMSO). The reaction solution was dialyzed at 4° C. against two 6 l changes of 20 mM HEPES pH 7.2 over 12 and 2 hours respectively. 1 ml of the derivatized and dialyzed Qβ solution was mixed with 4,400 μl gdHA_PR8_{—}42_{—}310 [0.5 mg/ml], 5,450 μl gdHA_PR8_{—}46_{—}310 [0.4 mg/ml], 2,090 μl gdHA_PR8_{—}54a_{—}276 [0.45 mg/ml], 2,000 μl gdHA_PR8_{—}57_{—}276 [0.45 mg/ml], 2,950 μl gdHA_PR8_{—}54a_{—}270 [0.6 mg/ml] and 3,529 μl gdHA_PR8_{—}57_{—}270 obtained from EXAMPLE 16 resulting in Qβ_gdHA_PR8_{—}42_{—}310, Qβ_gdHA_PR8_{—}46_{—}310, Qβ_gdHA_PR8_{—}54a_{—}276, Qβ_gdHA_PR8_{—}57_{—}276, Qβ_gdHA_PR8_{—}54a_{—}270. Non coupled proteins were removed by size exclusion chromatography using a Sepharose CL4B column. Coupled products were analyzed on a 4-12% Bis-Tris-polyacrylamide gel under reducing conditions. Several bands of increased molecular weight with respect to Qβ monomer and gdHA-PR8 monomers were visible, clearly demonstrating the successful cross-linking of all the globular domain fragments of PR8 to Qβ VLPs. A solution of 6 ml of 1 mg/ml AP205 capsid protein in 20 mM HEPES pH 7.2 will be reacted for 60 min at room temperature with 128 μl of a SMPH solution (50 mM in DMSO). The reaction solution was dialyzed at 4° C. against two 6 l changes of 20 mM HEPES pH 7.2 over 12 and 2 hours. 1 ml derivatized and dialyzed AP205 solution was mixed with 4,400 μl gdHA_PR8_{—}42_{—}310 [0.5 mg/ml], 5,450 μl gdHA_PR8_{—}46_{—}310 [0.4 mg/ml], 2,090 μl gdHA_PR8_{—}54a_{—}276 [0.45 mg/ml], 2,000 μl gdHA_PR8_{—}57_{—}276 [0.45 mg/ml], 2,950 μl gdHA_PR8_{—}54a_{—}270 [0.6 mg/ml] and 3,529 μl gdHA_PR8_{—}57_{—}270 resulting in AP205_gdHA_PR8_{—}42_{—}310, AP205_gdHA_PR8_{—}46_{—}310, AP205_gdHA_PR8_{—}54a_{—}276, AP205_gdHA_PR8_{—}57_{—}276, AP205_gdHA_PR8_{—}54a_{—}270, AP205_gdHA_PR8_{—}57_{—}270. Uncoupled protein was removed by size exclusion chromatography using a Sepharose CL4B column. Coupled products were analyzed on a 4-12% Bis-Tris-polyacrylamide gel under reducing conditions. Several bands of increased molecular weight with respect to the AP205 capsid monomer and gdHA-PR8 monomers were visible, clearly demonstrating the successful cross-linking of all the globular domain fragments of PR8 to AP205 VLPs.

### Example 26

### Efficacy Testing of Different gdHA Derived from ma A/PR/8/34

In order to test whether the different globular domain constructs generated from A/PR/8/34 in EXAMPLE 16 where able to induce a protective immune response, the vaccines generated with these globular domains obtained in EXAMPLE 25 were tested in an influenza mouse model. As a positive control a vaccine containing the whole extracellular domain (obtained from EXAMPLE 5, Qβ-ecHA(PR8) was used). Briefly, four female balb/c mice per group were immunized s.c. on day 0 with 15 μg of the antigens indicated in the first column of Table 7 formulated in 200 μl PBS. Mice were bled retro-orbitally on day 21 and sera were analyzed using ecHA(PR8)-specific ELISA as described in EXAMPLE 6 and hemagglutination inhibition (HAI) assay as described in EXAMPLE 7. To test the protective potential of the vaccine, all the mice were challenged with a lethal dose (10LD50) of influenza A/PR/8/34 on day 28 and the mice were monitored as described in EXAMPLE 8. The antibody titers, HAI titers as well as the survival after challenge are summarized in Table 7. Taken together these results show that most of the globular domains used for the production of vaccine showed higher titers when coupled to the bacteriophage VLPs as if the whole extracellular domain was coupled to the same VLP, strongly suggesting the fragment used contains the right epitopes and conformation. Moreover all the vaccines made with the globular domain fully protected mice from a lethal challenge with a homologous virus whilst most of the globular domains alone failed to protect mice from a lethal challenge further demonstrating that display on bacteriophage VLPs strongly enhances the immunogenicity of the antigens attached. Moreover upon coupling of the antigen to the VLPs the immune response against Qβ is strongly reduced which minimizes the risk of carrier induced epitopic suppression.

TABLE 7

| | Anti-ecHA-PR8- | Anti-Qβ-IgG | HAI titer | Survival [%] |
|---|---|---|---|---|
| Antigen | IgG d21 | d21 | d21 | 20 d p.i. |
| Q□_gdHA_PR8_42_310 | 17′053 | 15′760 | 53 | 100 |
| Q□_gdHA_PR8_46_310 | 15′242 | 19′088 | 40 | 100 |
| Q□_gdHA_PR8_54a_276 | 3′688 | 12′881 | 5 | 100 |
| Q□_gdHA_PR8_57_276 | 1′989 | 17′338 | 8 | 100 |
| Q□_gdHA_PR8_54a_270 | 1′103 | 20′814 | 0 | 100 |
| gdHA_PR8_42_310 | 119 | 11 | 0 | 25 |
| gdHA_PR8_46_310 | 336 | 11 | 0 | 100 |
| gdHA_PR8_54a_276 | 11 | 11 | 0 | 0 |
| gdHA_PR8_57_276 | 11 | 11 | 0 | 0 |
| gdHA_PR8_54a_270 | 11 | 11 | 0 | 0 |
| Qβ-ecHA(PR8) | 3′770 | 393 | 16 | 100 |
| ecHA(PR8) | 52 | 11 | 0 | 75 |
| Qβ | 11 | 165′327 | 0 | 0 |

### Example 27

### Protection Against Heterologous Virus Challenge

In order to get further insights into the protective potential of the vaccines based on gdHA six female balb/c mice per group were immunized s.c. on day 0 with 15 μg of Qβ_gdHA_PR8_{—}42_{—}310 or Qβ_gdHA_PR8_{—}46_{—}310 (obtained in EXAMPLE 16) or 15 ug of Qβ_ecHA(PR8) (obtained in EXAMPLE 5) or 15 μg of total protein of ecHA(PR8) (obtained in EXAMPLE 1) or 15 μg of total protein of Qβ formulated in 200 μl PBS. Mice were bled retro-orbitally on day 16 and sera were analyzed using ecHA(PR8)-specific ELISA as described in EXAMPLE 6 and hemagglutination inhibition (HAI) assay as described in Example 7. To test the protective potential of the vaccine, all mice were challenged with a lethal dose (10LD50) of the heterologous influenza A strains A/WSN/33 and A/FM/1/47 on day 23 and the mice were monitored as described in EXAMPLE 8. The antibody titers, HAI titers as well as the survival after challenge are summarized in Table 8. As shown in Table 8 the two globular domains of HA conjugated to Qβ VLPs induced high antibody titers against native HA derived from the homologous virus. Likewise good HAI titers against the homologous virus were induced. These antibody and HAI titers were similar or better than the ones induced by vaccine composed off the whole extracellular domain conjugated to the VLPs. It is important to note that both vaccine with globular domains fully protected mice from a heterologous challenge with two different H1N1 strains (A/FM/47 and A/WSN/33) whilst immunization with the complete native extracellular domain failed to provide full protection. This result further underscores the potential of the fragments of the extracellular domain chosen for the production of influenza vaccines.

TABLE 8

| | Challenge | Anti-ecHA-PR8- | Anti-Qβ-IgG | HAI titer | Survival [%] |
|---|---|---|---|---|---|
| Antigen | Strain | IgG d16 | d16 | d16 | 20 d p.i. |
| Qβ_gdHA_PR8_42_310 | A/WSN/33 | 7′135 | 14′234 | 36 | 100 |
| Qβ_gdHA_PR8_46_310 | | 4′563 | 16′957 | 15 | 100 |
| Qβ-ecHA(PR8) | | 1′207 | 204 | 16 | 100 |
| ecHA(PR8) | | 20 | 20 | 9 | 0 |
| Qβ | | 20 | 205′773 | 4 | 0 |
| Qβ_gdHA_PR8_42_310 | A/FM/1/47 | 10′446 | 12′438 | 45 | 100 |
| Qβ_gdHA_PR8_46_310 | | 6′425 | 21′119 | 31 | 100 |
| Qβ-ecHA(PR8) | | 1′637 | 280 | 12 | 100 |
| ecHA(PR8) | | 20 | 20 | 7 | 33.3 |
| Qβ | | 20 | 202′316 | 7 | 0 |

### Example 28

### Dose Titration of Globular Domains Conjugated to Qβ

In order to get more insights into the protective potential of the vaccine four female balb/c mice per group were immunized s.c. on day 0 with 15, 3, 0.6, 0.12, 0.024 or 0.0046 μg of gdHA_PR8_{—}42_{—}310 or gdHA_PR8_{—}46_{—}310 conjugated to Qβ (obtained from EXAMPLE 16) or 15 μg of ecHA(PR8) (obtained in EXAMPLE 1) or 15 μg of Qβ in 200 μl PBS (see also first two rows of Table 9). Mice were bled retro-orbitally on day 18 and sera were analyzed using ecHA(PR8)-specific ELISA or hemagglutination inhibition (HAI) assay as described in EXAMPLES 6 and 7 respectively. To test the protective potential of the vaccine, all the mice were challenged with a lethal dose (4LD50) of influenza A/PR/8/34 on day 21 and the mice were monitored as described in EXAMPLE 8. The antibody Titers, HAI titers as well as the survival after challenge are summarized in Table 9. As shown in Table 9 vaccines with both globular domains investigated induced high antibody titers against the native HA from the homologous virus and good HAI titers determined with the homologous virus strain. Moreover, a single injection with 120 ng of vaccine was able to fully protect mice from a lethal challenge with the homologous virus. It is important to note that 15 μg of the extracellular domain of HA produced in a eukaryotic expression system was not able to fully protect mice from a lethal challenge, further highlighting the potency of the vaccines based on the globular domain of HA. Like observed above, coupling of antigens to the bacteriophage VLP strongly reduces the carrier specific immune response.

TABLE 9

| | Amount | Anti-ecHA-PR8- | Anti-Qβ-IgG | HAI titer | |
|---|---|---|---|---|---|
| Antigen | [μg] | IgG d18 | d18 | d18 | Survival [%] |
| Qβ_gdHA_PR8_42_310 | 15 | 24′469 | 15′479 | 128 | 100 |
| | 3 | 4′758 | 5′469 | 36 | 100 |
| | 0.6 | 3′703 | 3′986 | 25 | 100 |
| | 0.12 | 2′048 | 2′556 | 20 | 100 |
| | 0.024 | 210 | 454 | 14 | 50 |
| | 0.0046 | 253 | 300 | 16 | 100 |
| Qβ_gdHA_PR8_46_310 | 15 | 9′138 | 16′414 | 52 | 100 |
| | 3 | 4′182 | 5′684 | 36 | 100 |
| | 0.6 | 1′838 | 2′635 | 16 | 100 |
| | 0.12 | 913 | 655 | 16 | 100 |
| | 0.024 | 176 | 753 | 14 | 75 |
| | 0.0046 | 383 | 352 | 10 | 50 |
| ecHA(PR8) | 15 | 25 | 20 | 4 | 50 |
| Qβ | 15 | 20 | 135532 | 8 | 0 |

### Example 29

### Dose Titration of Globular Domains Conjugated to AP205

In order to get more insights into the protective potential of the vaccine four female balb/c mice per group were immunized s.c. on day 0 with 15, 3, 0.6, 0.12, 0.024 or 0.0046 μg of gdHA_PR8_{—}42_{—}310 or gdHA_PR8_{—}46_{—}310 conjugated to AP205 (obtained from EXAMPLE 16) or 15 μg of ecHA(PR8) (obtained in EXAMPLE 1) or 15 μg of AP205 in 200 μl PBS (see also first two rows of Table 10). Mice were bled retro-orbitally on day 21 and sera were analyzed using ecHA(PR8)-specific ELISA or hemagglutination inhibition (HAI) assay as described in EXAMPLES 6 and 7 respectively. To test the protective potential of the vaccine, all the mice were challenged with a lethal dose (4LD50) of influenza A/PR/8/34 on day 34 and the mice were monitored as described in EXAMPLE 8. The antibody Titers, HAI titers as well as the survival after challenge are summarized in Table 10. As shown in Table 10 vaccines with both globular domains investigated induced high antibody titers against the native HA from the homologous virus and good HAI titers determined with the homologous virus strain. Moreover, a single injection with 24 ng or 120 ng vaccine (depending on the globular domain used) was able to fully protect mice from a lethal challenge with the homologous virus, further highlighting the potency of the vaccines based on the globular domain of HA. Like observed above, coupling of antigens to the bacteriophage VLP strongly reduces the carrier specific immune response.

TABLE 10

| | | Anti-ecHA-PR8- | Anti-AP205- | HAI titer | Survival [%] |
|---|---|---|---|---|---|
| Antigen | Amount [μg] | IgG d21 | IgG d21 | d21 | 20 d p.i |
| AP205_gdHA_PR8_42_310 | 15 | 4′065 | 2′512 | 53 | 100 |
| | 3 | 2′718 | 1′930 | 16 | 100 |
| | 0.6 | 2′029 | 384 | 18 | 100 |
| | 0.12 | 2′590 | 1′344 | 11 | 100 |
| | 0.024 | 2′040 | 549 | 10 | 100 |
| | 0.0046 | 36 | 77 | 8 | 50 |
| AP205_gdHA_PR8_46_310 | 15 | 4′595 | 4′411 | 29 | 100 |
| | 3 | 4′763 | 3′582 | 28 | 100 |
| | 0.6 | 1′235 | 986 | 13 | 100 |
| | 0.12 | 2′293 | 559 | 16 | 100 |
| | 0.024 | 392 | 368 | 11 | 50 |
| | 0.0046 | 333 | 340 | 12 | 100 |
| ecHA(PR8) | 15 | 699 | 20 | 18 | 100 |
| AP205 | 15 | n.d. | n.d. | 9 | 0 |

### Example 30

### Immunisation with gdHA_PR8_{—}42_{—}310 and gdHA_PR8_{—}46_{—}310 Conjugated to Bacteriophage VLPs+/−Alum, +/−Boost

In order to further explore the immunogenicity of the vaccine in conjunction with an adjuvant, four female balb/c mice per group were immunized s.c. on days 0 and 24 with 15, 3, 0.6 or 0.12 μg of Qβ_gdHA_PR8_{—}42_{—}310, Qβ_gdHA_PR8_{—}46_{—}310, AP205_gdHA_PR8_{—}42_{—}310 or AP205_gdHA_PR8_{—}46_{—}310 (obtained in EXAMPLE 16) with or without Alum (8.3 μl Alhydrogel 2% (Brenntag, Biosector) per mouse per injection) per mouse per injection) formulated in 200 μl PBS. Mice were bled retro-orbitally on day 24 and day 48 and sera were analyzed using ecHA(PR8)-specific ELISA or hemagglutination inhibition (HAI) assay. The average anti-ecHA-PR8 antibody titers at day 24 and day 48 are shown in Table 11. The results in Table 11 demonstrate that all vaccine induced good antibody responses against the native extracellular domain of the homologous virus at each concentration tested. The same is true for HAI titers. The initial titers (ELISA and HAI) could be significantly boosted by a second injection with the same dose of vaccine. Moreover the data show that the addition of alum to the vaccine even further increased the immune response induced.

TABLE 11

| | | Anti-ecHA- | Anti-ecHA- | HAI | HAI titer |
|---|---|---|---|---|---|
| Antigen | Amount [μg] | PR8-IgG d24 | PR8-IgG d48 | titer, d24 | d48 |
| Qβ_gdHA_PR8_42_310 | 15 | 13′459 | 93′686 | 144 | 832 |
| | 3 | 7′038 | 63′480 | 112 | 608 |
| | 0.6 | 2′664 | 33′886 | 104 | 320 |
| | 0.12 | 2′697 | 44′372 | 128 | 160 |
| Qβ_gdHA_PR8_42_310 + | 15 | 52′750 | 269′884 | 576 | 2′944 |
| Alum | 3 | 24′250 | 169′454 | 108 | 1′664 |
| | 0.6 | 10′500 | 334′500 | 52 | 2′496 |
| | 0.12 | 8′305 | 125′812 | 52 | 1′160 |
| AP205_gdHA_PR8_42_310 | 15 | 5′625 | 58′828 | 30 | 992 |
| | 3 | 3′208 | 40′477 | 26 | 328 |
| | 0.6 | 2′868 | 63′254 | 30 | 768 |
| | 0.12 | 1′225 | 34′125 | 26 | 240 |
| AP205_gdHA_PR8_42_310 + | 15 | 26′833 | 236′884 | 172 | 2′816 |
| Alum | 3 | 11′491 | 327′045 | 64 | 2′368 |
| | 0.6 | 4′499 | 153′183 | 52 | 1′800 |
| | 0.12 | 3′774 | 53′321 | 24 | 198 |

### Example 31

### Efficacy of a Vaccine Consisting of the Globular Domain of A/California/04/09 Coupled to Bacteriophage VLPs

In order to test the globular domain from influenza A/California/04/2009 (H1N1) a vaccine was produced and tested in a mouse efficacy study with a heterologous virus challenge. Briefly, the globular domain from influenza A A/California/04/2009 (obtained in EXAMPLE 18) was coupled to Qβ and AP205 and uncoupled proteins removed, essentially as described in EXAMPLE 25. The resulting vaccines were named Qβ_gdHA_AC0409_{—}42_{—}310 and AP205_gdHA_AC0409_{—}42_{—}310. Four female balb/c mice per group were immunized s.c. and day 0 and day 28 with 75, 15, 3, 0.6 or 0.12 μg of Qβ_gdHA_AC0409_{—}42_{—}310 or AP205_gdHA_AC0409_{—}42_{—}310 with or without Alum (8.3 μl Alhydrogel 2% (Brenntag, Biosector) per mouse per injection) formulated in 200 μl PBS. Mice were bled retro-orbitally on day 21 and day 49 and sera were analyzed using rHA(A/California/04/09)-specific ELISA as described in EXAMPLE 6. At day 65 Mice were challenged with a lethal dose of 4LD50 of a heterologous mouse adapted influenza A/PR/8/34 virus and the mice were monitored for survival as described in EXAMPLE 8. The results of this experiment are summarized in Table 12. The results shown in Table 12 demonstrates that IgG antibodies induced by immunization of mice with a variant of the ectodomain of influenza A/California/04/09 virus hemagglutinin, which was expressed in E. coli and refolded, recognize the native trimeric form of the influenza A/California/04/09 Hemagglutinin protein. Both vaccines induced good antibody responses against the native extracellular domain of the homologous virus at each concentration tested. The initial titers could be significantly boosted by a second injection with the same dose of vaccine. Moreover the data show that the addition of alum to the vaccine even further increased the immune response against the coupled antigen. Importantly with the exception of one experimental group all mice which had been immunized with the globular domain coupled to bacteriophage VLPs, whether administered alone or together with alum, survived the lethal challenge with a heterologous virus. In stark contrast only partial protection was observed if 15 μg of the globular domain alone were administered together with alum. Likewise all animals which had received the globular domain alone without alum died. Taken together these results further demonstrate that coupling of the globular domain to bacteriophage VLP significantly improves its protective potential.

TABLE 12

| | | Anti- | Anti- | |
|---|---|---|---|---|
| | | rHA_AC0409- | rHA_AC0409- | Survival [%] |
| Antigen | Amount [μg] | IgG, d21 | IgG, d49 | 20 d p.i. |
| Qβ_gdHA_AC0409_42_310 | 75 | 11′135 | 228′833 | 100 |
| | 15 | 6′659 | 81′367 | 100 |
| | 3 | 1′609 | 43′685 | 100 |
| | 0.6 | 1′261 | 16′279 | 100 |
| | 0.12 | 2′156 | 42′705 | 100 |
| Qβ_gdHA_AC0409_42_310 + | 75 | 32′795 | 1′512085 | 100 |
| Alum | 15 | 15′275 | 301′255 | 100 |
| | 3 | 14′359 | 273′799 | 100 |
| | 0.6 | 5′672 | 112′484 | 100 |
| | 0.12 | 4′610 | 74′160 | 75 |
| AP205_gdHA_AC0409_42_310 | 75 | 5′344 | 319′694 | 100 |
| | 15 | 880 | 48′092 | 100 |
| | 3 | 603 | 15′382 | 100 |
| | 0.6 | 1′872 | 18′658 | 100 |
| | 0.12 | 744 | 29′731 | 100 |
| AP205_gdHA_AC0409_42_310 + | 75 | 22′543 | 538′403 | 100 |
| Alum | 15 | 17′448 | 435′710 | 100 |
| | 3 | 4′302 | 179′476 | 100 |
| | 0.6 | 6′039 | 207′914 | 100 |
| | 0.12 | 1′790 | 69′734 | 100 |
| gdHA_AC0409_42_310 | 75 | 20 | 2′505 | 0 |
| | 15 | 20 | 20 | 0 |
| gdHA_AC0409_42_310 + | 75 | 3′239 | 116′060 | 50 |
| Alum | 15 | 880 | 91′868 | 75 |
| Qβ | 15 | 20 | 20 | 25 |
| Qβ + Alum | 15 | 20 | 20 | 0 |
| AP205 | 15 | 20 | 20 | 0 |
| AP205 + Alum | 15 | 20 | 20 | 0 |

### Example 32

### Immunogenicity of gdHA from Different Influenza Strains in Mice

In order to test whether the globular domains from different influenza subtype can be used to generate vaccines which recognize native HA of the respective subtype vaccines with the globular domain of the different subtypes were generated and tested for their immunogenicity in mice. Briefly, the globular domain from influenza A H1N1 (obtained in EXAMPLE 19 and EXAMPLE 24), the globular domain of influenza A H3N2 (obtained in EXAMPLE 20), the globular domains from influenza A H5N1 strains (obtained in EXAMPLE 21 and 22) and the globular domain of influenza B (obtained in EXAMPLE 23) were coupled to Qβ and/or AP205 and uncoupled proteins removed, essentially as described in EXAMPLE 25. The resulting vaccines were named according to the VLP (Qβ or AP205) and the globular domain linked (e.g. Qβ_gdHA_AB5907_{—}42_{—}310). Three to five female balb/c mice per group were immunized once s.c. on day 0 with 15 μg of the antigen indicated in the first column of Table 13 formulated in 200 μl PBS. Mice were bled retro-orbitally on day 21 and sera were analyzed using HA specific ELISAs as described in EXAMPLE 6 using the coating indicated in the second column of Table 13. As shown in Table 13, the globular domains of all different influenza A subtypes (H1, H5 and H3) and the influenza B strain tested were able to elicit an antibody response which recognizes native HA from the respective influenza subtype. In each case coupling of gdHA domains to VLPs clearly increased their Immunogenicity compared to immunization with the gdHA alone. Importantly, the fact that the approach worked for all strains and subtypes investigated, strongly suggests that the globular domains which will work as vaccines can be predicted for future emerging influenza strains and subtypes.

TABLE 13

| | Mice per | Coating used for | Anti-HA IgG |
|---|---|---|---|
| Antigen | group | ELISA | titers d21 |
| Qβ_gdHA_AB5907_42_310 | 5 | rHA_A/Brisbane/59/2007 | 394 |
| AP205_gdHA_AB5907_42_310 | | | 30 |
| gdHA_AB5907_42_310 | | | 0 |
| AP205_gdHA_AU71607_42_310 | | ecHA-Uruguay | 26 |
| gdHA_AU71607_42_310 | | | 0 |
| AP205_gdHA_BB307_42_310 | | rHA_B/Florida/04/2006 | 476 |
| gdHA_BB307_42_310 | | | 33 |
| Qβ_gdHA_AV120304_42_310 | | rHA_A/Vietnam/1203/2004 | 92 |
| gdHA_AV120304_42_310 | | | 0 |
| Qβ_gdHA_AI505_42_310 | | rHA_A/Indonesia/05/2005 | 1058 |
| AP205_gdHA_AI505_42_310 | | | 49 |
| gdHA_AI505_42_310 | | | 0 |
| Qβ_gdHA_AC0709_42_310 | 3 | rHA_A/Califomia/04/2009 | 1334 |
| gdHA_AC0709_42_310 | | | 20 |

### Example 34

### Cb5

A) Coupling of gdHA_PR8_{—}42_{—}310 (H1N1) to Cb5 Virus-Like Particles

A solution of 2 ml of 1 mg/ml Cb5 VLPs protein (SEQ ID NO:92) in PBS/10% glycerol pH 7.2 was reacted for 60 min at room temperature with 42.6 μl of a SMPH solution (50 mM in DMSO). The reaction solution was dialyzed at 4° C. against two 2 l changes of 20 mM HEPES/10% glycerol pH 7.2 over 12 and 4 hours. 1.4 ml of the derivatized and dialyzed Cb5 solution was mixed with 2 ml of a solution containing 1 mg/ml of the purified gdHA_PR8_{—}42_{—}310 protein obtained in EXAMPLE 16 in PBS pH 7.2 and incubated 4 h at room temperature for chemical cross linking, resulting in Cb5-gdHA_PR8_{—}42_{—}310. Uncoupled protein was removed by size exclusion chromatography using a Sepharose CL4B column. The coupled product was analyzed on a 12% Bis-Tris-polyacrylamide gel under reducing conditions. A band of increased molecular weight with respect to the Cb5 capsid monomer was visible, clearly demonstrating the successful cross-linking of the influenza gdHA_PR8_{—}42_{—}310 protein to the Cb5 VLP.

B) Immunization of Mice with gdHA-PR8 (H1N1) Protein Coupled to Cb5 Capsids (Cb5-gdHA(PR8)

The efficacy of Cb5-gdHA(PR8) immunization was tested in a murine model of influenza infection as described in EXAMPLE 8. Briefly four female balb/c mice per group were immunized with 15 μg of Cb5-gdHA_PR8_{—}42_{—}310 vaccine or 15 μg of Cb5 VLPs formulated in 200 μl PBS and injected subcutaneously on day 0. Mice were bled retro-orbitally on day 34 and sera were analyzed using ecHA PR8-specific and Cb5-specific ELISA. Mice were then challenged at day 41 with a lethal dose (4×LD50) of mouse adapted influenza A/PR/8/34. The result of this experiment is shown in Table 14. The result shown in Table 14 demonstrates that coupling of gdHA(PR8) to Cb5 VLPs allows the induction of a high anti-ecHA(PR8) antibody response. Moreover a single immunization of mice with Cb5-gdHA(PR8) vaccine induces of a protective antibody response against a lethal challenge with mouse adapted influenza A/PR/8/34 demonstrating that Cb5 is a good carrier for influenza vaccines based on the globular domain of HA.

TABLE 14

| | | | | Survival |
|---|---|---|---|---|
| | Amount | | | [%] |
| Antigen | [μg] | Anti-ecHA(PR8) d34 | Anti-Cb5 | 20 d p.I |
| Cb5-gdHA(PR8) | 15 | 3′560 | 13′044 | 100 |
| Cb5 | 15 | n.d. | 10′510 | 0 |

### Example 35

### Hemagglutination Assay

In order to test if the gdHA fragments produced as described in Example 24 and coupled to Qβ or AP205 as described in Example 25 are structurally similar to native HA protein, a hemagglutination assay was performed with gdHA_PR8_{—}42_{—}310 or gdHA_PR8_{—}46_{—}310 conjugated to Qβ or AP205. Native HA proteins present on influenza viruses are able to agglutinate red blood cells as a consequence of their binding to their receptor on red blood cells (RBCs). This agglutination of chicken RBCs by influenza virus is inhibited in the hemagglutination inhibition assay by neutralizing antibodies as described in Example 7. To test if the gdHA fragments coupled to Qβ or AP205 had a similar structure as native HA protein on the surface of influenza viruses and therefore were able to bind to the receptor on RBCs and as consequence were inducing agglutination of chicken RBCs, Qβ-gdHA_PR8_{—}42_{—}310, Qβ-gdHA_PR8_{—}46_{—}310, AP205-gdHA_PR8_{—}42_{—}310 and AP205-gdHA_PR8_{—}46_{—}310 solutions were serially diluted in PBS and mixed with 50 μl of 1% chicken RBCs in 96 well plates. The plates were mixed by agitation, covered, and the RBCs were allowed to settle for 1 h at room temperature. The minimal amount of Qβ-gdHA_PR8_{—}42_{—}310, Qβ-gdHA_PR8_{—}46_{—}310, AP205-gdHA_PR8_{—}42_{—}310 and AP205-gdHA_PR8_{—}46_{—}310 which were still able to agglutinate the chicken RBCs was determined and was 80 ng/well for Qβ-gdHA_PR8_{—}42_{—}310, 80 ng/well for Qβ-gdHA_PR8_{—}42_{—}310, 40 ng/well for AP205-gdHA_PR8_{—}42_{—}310 and 10 ng/well for AP205-gdHA_PR8_{—}46_{—}310. The result of this experiment shows that fragments of gdHA can bind to the receptor of the native HA protein and therefore must be structurally similar to native HA protein.

1. A composition comprising:
(a) a virus-like particle (VLP) with at least one first attachment site, wherein said virus-like particle is a virus-like particle of an RNA bacteriophage; and
(b) at least one antigen with at least one second attachment site, wherein said at least one antigen is a fragment of an said ectodomain of an influenza virus hemagglutinin protein, wherein said fragment of said ectodomain of an influenza virus hemagglutinin protein comprises at least 80 contiguous amino acids of said ectodomain of an influenza virus hemagglutinin protein, and wherein said ectodomain of an influenza virus hemagglutinin protein is an ectodomain of an influenza A virus hemagglutinin protein;
wherein (a) and (b) are linked through said at least one first and said at least one second attachment site, and wherein said first attachment site and said second attachment site are linked via at least one non-peptide covalent bond.

2. (canceled)

3. (canceled)

4. The composition of claim 1, wherein said ectodomain of said influenza A virus hemagglutinin protein is an ectodomain of an influenza A virus hemagglutinin protein subtype selected from the group consisting of influenza A virus hemagglutinin protein subtype H1, H2, H3, H4, H5, H6, H7, H8, H9, H10, H11, H12, H13, H14, H15, and H16.

5. (canceled)

6. The composition of claim 1, wherein the amino acid sequence of said ectodomain of said influenza A virus hemagglutinin protein is selected from the group consisting of: (i) the amino acid sequence as set forth in SEQ ID NO:39; and (ii) an amino acid sequence of at least 70% amino acid sequence identity with SEQ ID NO:39.

7. The composition of claim 1, wherein the amino acid sequence of said ectodomain of said influenza A virus hemagglutinin protein is selected from the group consisting of:
(i) the amino acid sequence as set forth in SEQ ID NO:40; and
(ii) the amino acid sequence as set forth in SEQ ID NO:41:
(iii) the amino acid sequence as set forth in SEQ ID NO:42:
(iv) the amino acid sequence as set forth in SEQ ID NO:43:
(v) the amino acid sequence as set forth in SEQ ID NO:73: and
(vi) an amino acid sequence of at least 70% amino acid sequence identity with any one of SEQ ID NO:40. SEQ ID NO:41, SEQ ID NO:42. SEQ ID NO:43 or SEQ ID NO:73.

8.-11. (canceled)

12. The composition of claim 1, wherein the amino acid sequence of said ectodomain of said influenza A virus hemagglutinin protein is selected from the group consisting of: (i) the amino acid sequence as set forth in SEQ ID NO:74; and (ii) an amino acid sequence of at least 70% amino acid sequence identity with SEQ ID NO:74.

13. (canceled)

14. (canceled)

15. The composition of claim 1, wherein said fragment of said ectodomain of an influenza virus hemagglutinin protein is the HA1 subunit of said ectodomain of an influenza virus hemagglutinin protein or a fragment of said HA1 subunit of said ectodomain of an influenza virus hemagglutinin protein.

16.-18. (canceled)

19. The composition of claim 15, wherein said fragment of said ectodomain of an influenza virus hemagglutinin protein comprises the amino acid residues tyrosine corresponding to the positions 98 and 195 of SEQ ID NO:75, tryptophan corresponding to the position 153 of SEQ ID NO:75, and histidine corresponding to of SEQ ID NO:75.

20. The composition of claim 15, wherein said fragment of said ectodomain of an influenza virus hemagglutinin protein comprises a cysteine residue corresponding to positions 52, 64, 76, 97, 139, 277, 281, 305 of SEQ ID NO:75.

21. The composition of claim 15, wherein said fragment of said ectodomain of an influenza virus hemagglutinin protein comprises or consists of:
(i) an amino acid sequence corresponding to position 57 to position 276 of SEQ ID NO:75;
(ii) an amino acid sequence corresponding to position 54 to position 276 of SEQ ID NO:75;
(iii) an amino acid sequence corresponding to position 54 to position 270 of SEQ ID NO:75
(iv) an amino acid sequence corresponding to position 54a to position 276 of SEQ ID NO:75:
(v) an amino acid sequence corresponding to position 54a to position 270 of SEQ ID NO:75.

22. The composition of claim 15, wherein said fragment of said ectodomain of an influenza virus hemagglutinin protein comprises or consists of an amino acid sequence corresponding to position 46 to position 310 of SEQ ID NO:75.

23. The composition of claim 15, wherein said fragment of said ectodomain of an influenza virus hemagglutinin protein comprises or consists of an amino acid sequence corresponding to position 42 to position 310 of SEQ ID NO:75.

24.-27. (canceled)

28. The composition of claim 15, wherein said ectodomain of an influenza virus hemagglutinin protein has an amino acid sequence identity of at least 70% with the HA ectodomain of influenza A virus strain A/California/07/2009 (H1N1) (Genebank Accession No: ACP44189.1) or A/Perth/16/2009 (H3N2) (Genebank Accession No: ACS71642.1).

29.-32. (canceled)

33. The composition claim 1, wherein said VLP is a VLP of RNA bacteriophage Qβ.

34. The composition of claim 1, wherein said virus-like particle comprises recombinant coat proteins of RNA bacteriophage Qβ, wherein said recombinant coat proteins comprise the amino acid sequence of SEQ ID NO:1.

35. (canceled)

36. (canceled)

37. The composition of claim 1, wherein said first attachment site is an amino group of a lysine residue, and wherein said second attachment site is a sulfhydryl group of a cysteine residue.

38. (canceled)

39. A vaccine composition comprising an effective amount of the composition of claim 1.

40. A pharmaceutical composition comprising:
(a) the composition of claim 1 or the vaccine composition of claim 39; and
(b) a pharmaceutically acceptable carrier.

41. A method of immunization, said method comprising administering the composition of claim 1 or the vaccine composition of claim 39 to a human.

42. (canceled)

43. (canceled)

44. A method of treatment, amelioration and/or prevention of influenza said method comprising administering an immunologically effective amount of the composition of claim 1 or the vaccine composition of claim 39 to a human.

45. The composition of claim 1, wherein said virus-like particle comprises recombinant coat proteins of RNA bacteriophage Qβ consisting of SEQ ID NO:1.
